(19) United States
(12) Patent Application Publication
(10) Pub. No.: US 2011/0306671 A1
(43) Pub. Date: Dec. 15, 2011

(54) AMIDE DERIVATIVES OF ETHACRYNIC ACID

(75) Inventors: Dennis A. Carson, La Jolla, CA (US); Howard B. Cottam, Escondido, CA (US); Guangyi Jin, Berkeley, CA (US); Desheng Lu, San Diego, CA (US)

(73) Assignee: The Regents of the The University of California, Oakland, CA (US)

(21) Appl. No.: 13/140,613

(22) PCT Filed: Dec. 16, 2009
(86) PCT No.: PCT/US09/06584

§ 371 Date: Sep. 1, 2011

Related U.S. Application Data
(60) Provisional application No. 61/138,381, filed on Dec. 17, 2008.

(51) Int. Cl. A61K 31/165; A61P 35/00; A61P 35/02; C07C 233/01

(52) U.S. Cl. 514/621; 564/169

(57) ABSTRACT

The invention provides ethacrynic acid derivatives useful to prevent, inhibit or treat a variety of disorders or diseases including cancer and inflammatory disorders.

### CROSS-REFERENCE TO RELATED APPLICATIONS

This application claims the benefit of the filing date of U.S. application Ser. No. 61/138,381, filed on Dec. 17, 2008, the disclosure of which is incorporated herein by reference in its entirety.

### STATEMENT OF GOVERNMENT RIGHTS

The invention was made with Government support under CA113318 awarded by the National Institutes of Health. The Government has certain rights in the invention.

### BACKGROUND

Chronic lymphocytic leukemia (CLL), the most common adult leukemia in the United States, is characterized by the accumulation of mature-appearing, but functionally incompetent small lymphocytes. There is as yet no cure for this disease, nor has conventional chemotherapy been definitively shown to prolong patient survival. Late in the disease course, patients typically develop pronounced bone marrow dysfunction due to chemotherapy-induced toxicity and disease progression, making them intolerant to further treatment with cytotoxic agents. Thus, it is necessary to develop new treatments that target the molecular defects in CLL with minimal bone marrow toxicities. The clonal expansion of B lymphocytes in CLL is caused by an abnormal balance between the signaling for survival and cell death (Caligaris-Cappio et al., 1999).

Wnt signaling pathways play a number of key roles in embryonic development and maintenance of homeostasis in mature tissues. Wnt proteins are a large family of secreted glycoproteins that activate signal transduction pathways to control a wide variety of cellular processes such as determination of cell fate, proliferation, migration, and polarity. Wnts are capable of signaling through several pathways, the best-characterized being the canonical β-catenin/Tcf-LEF mediated pathway. Canonical Wnts stabilize β-catenin protein, which has implications in the genesis of many human cancers. Indeed, growing evidence suggests that deregulation of the Wnt/□-catenin pathway is directly linked to tumorigenesis (Peifer et al., 2000; Polakis, 2000).

### SUMMARY OF THE INVENTION

Ethacrynic acid (EA) kills chronic lymphocytic leukemia (CLL) cells at a lower dose than that required to kill normal (noncancerous) B cells. However, it is a diuretic and patients on this medication need to be given fluids to maintain hydration. As described herein, compounds that are amide derivatives of ethacrynic acid, an approved drug used as a loop diuretic, were prepared and evaluated for inhibition of Wnt signaling and/or reduction in the survival of CLL cells. The preparation of these compounds is accomplished by using standard amide formation reactions starting from the free carboxylic acid, such as ethacrynic acid. For example, the acid, ethacrynic acid, can be converted to the acid chloride by treatment with thionyl chloride and then reacted with the appropriate amine to form the desired amide as the final compound. Several of the most potent derivatives were active in the low micromolar range. Reduction of the □□-unsaturated carbon-carbon double bond of EA abrogated both the inhibition of Wnt signaling as well as the decrease in CLL survival. These derivatives may covalently modify sulfhydryl groups present on transcription factors important for Wnt/β-catenin signaling. The derivatives may also inhibit NF-kB and so be useful to prevent, inhibit or treat inflammatory disorders. Moreover, the compounds of the invention may have reduced diuretic activity, e.g., they are not diuretics, and may be more potent compounds for the killing of CLL cells than is ethacrynic acid.

In one embodiment, ethacrynic amide compounds are provided. The ethacrynic amides described herein can be used in methods to inhibit or treat cancer in a mammal. Such methods can include administering to a mammal in need thereof an effective amount of a composition comprising an amide of ethacrynic acid, so that the cancer is thereby inhibited or treated. The amide of ethacrynic acid can be, for example, a hydroxyl amide of ethacrynic acid, or an optionally substituted alkyl amide, aryl amide, heteroaryl amide, or heterocycle amide of ethacrynic acid, wherein the optional substitution of the amide moiety is as described or illustrated herein.

In one embodiment, the ethacrynic derivatives are effective in controlling the growth and/or survival of certain cancer cells, particularly hematopoietic cancer cells, such as cancerous B cells, for instance, CLL cells. In one embodiment, compounds, e.g., those shown in Table 1, are effective at inhibiting, e.g., killing, cancerous B cells, such as CLL cells, at a lower dose than that required to inhibit, e.g., kill, normal human B cells. In one embodiment, derivatives of ethacrynic acid useful in the methods of the invention have reduced, e.g., a reduction of 30%, 40%, 50%, 70%, 90% or more, or no, diuretic activity relative to ethacrynic acid, and therefore are more suitable than ethacrynic acid for treatment of cancer patients. In one embodiment, the derivatives are more potent and have reduced or no diuretic activity.

In one embodiment, the ethacrynic derivatives of the invention are useful to inhibit or treat chronic or acute leukemia, including chronic or acute myelogenous leukemia (lymphoma) or chronic or acute lymphocytic leukemia, including but not limited to CLL, acute myelogenous leukemia (AML), acute lymphocytic leukemia (ALL), non-Hodgkin's lymphoma, follicular lymphoma, anaplastic large cell lymphoma, Burkitts' and Burkitt-like lymphoma, hairy cell leukemia, Hodgkin's lymphoma, and AIDS-related lymphoma. In one embodiment, compounds, e.g., those shown in Table 1, are effective to inhibit or treat leukemias at a lower dose than that required to inhibit, e.g., kill, corresponding normal cells. In one embodiment, derivatives of ethacrynic acid useful in the methods of the invention have reduced, e.g., a reduction of 30%, 40%, 50%, 70%, 90% or more, or no, diuretic activity relative to ethacrynic acid, and therefore are more suitable than ethacrynic acid for treatment of cancer patients. In one embodiment, the derivatives are more potent and have reduced or no diuretic activity.

In one embodiment, the ethacrynic derivatives of the invention are useful to inhibit or treat solid tumors, e.g., sarcomas and carcinomas inclduign breast and prostate cancer. In one embodiment, compounds, e.g., those shown in Table 1, are effective at inhibiting, e.g., killing, solid tumor cells at a lower dose than that required to inhibit, e.g., kill, corresponding normal cells. In one embodiment, derivatives of ethacrynic acid useful in the methods of the invention have reduced, e.g., a reduction of 30%, 40%, 50%, 70%, 90% or more, or no, diuretic activity relative to ethacrynic acid, and therefore are more suitable than ethacrynic acid for treatment of cancer patients. In one embodiment, the derivatives are more potent and have reduced or no diuretic activity.

In one embodiment, the ethacrynic derivatives of the invention are effective to prevent, inhibit or treat a disease or disorder associated with NF-kB, e.g., aberrant NF-kB expression or activity. In one embodiment, a compound of formula (I) is employed to prevent, inhibit or treat an inflammatory disorder associated with NF-kB. Exemplary disorders associated with NF-kB include but are not limited to allergies, headache, cardiac hypertrophy, atherosclerosis, ischemia/reperfusion, stroke, cystic fibrosis, hypertension, e.g., pulmonary hypertension, kidney disease, glomerular disease, intestinal disease, sinusitis, asthma, arthritis, Crohn's disease, inflammatory bowel disease, Lupus or other autoimmune disorders such as multiple sclerosis, chronic disease syndrome, or Parkinson disease.

In one embodiment, the derivatives of the invention contain an □\tilde{□}unsaturated carbonyl function which allows for addition of certain nucleophiles, particularly thiols, such as glutathione and other cysteine-containing peptides and proteins. Thus, these compounds become, in a sense, alkylators of thiol-containing peptides and proteins. Some of these peptides and proteins, such as LEF-1 and IKK-beta, appear to be essential for normal growth and survival of some cancer cells, including CLL cells, and may be covalently modified by this Michael-type addition to the compounds.

In one embodiment, the invention provides a method to mediate killing of tumor cells in a mammal in need of such therapy. The method includes administering an effective amount of at least one compound of the invention to the mammal, e.g., a human. In one embodiment, the at least one compound is intravenously administered. In one embodiment, the compound is orally administered, e.g., in tablet form. In one embodiment, the compound is administered in conjunction with another chemotherapeutic agent, e.g., concurrently or sequentially, or another anti-cancer therapy, such as radiation.

The present invention also provides a method for inhibiting or eliminating tumor cells. In one embodiment, cells are contacted with at least one compound of the invention, e.g., ex vivo.

Further provided is a method of inhibiting metastases. The method includes administering to mammal having cancer an effective amount of at least one compound of the invention.

In one embodiment, an ethacrynic derivative of the invention is useful to inhibit the proliferation or survival or kill cancer stem cells, e.g., cancerous hematopoietic stem cells such as those for CLL. In one embodiment, a cancer stem cell in a mammal having cancer is identified and an ethacrynic acid derivative useful to inhibit the proliferation or survival of that cancer stem cell is selected for administration to that mammal. In one embodiment, an ethacrynic derivative of the invention is useful to sensitize cancer stem cells, e.g., cancerous hematopoietic stem cells, to other anti-cancer therapies, e.g., chemotherapeutics or radiation therapy. For instance, stem cells for acute myelogenous leukemia (AML) may be CD34⁺ CD38⁻ cells and a ethacrynic derivative of the invention may inhibit those cells or sensitize those cells to anti-AML treatments such as cytarabine and an anthracycline drug such as daunorubicin (daunomycin) or idarubicin. Hematopoietic cancer stem cells which may be inhibited or killed by a compound of the invention include cells that are Thy-1⁻, c-kit⁻, and IL-3R-alpha⁺.

In one embodiment, an ethacrynic derivative of the invention is useful to inhibit the proliferation or survival or kill solid tumor stem cells, e.g., cancerous pancreatic, liver, colorectal, breast or prostate stem cells. In one embodiment, a cancer stem cell in a mammal having a solid cancer is identified and an ethacrynic acid derivative useful to inhibit the proliferation or survival of that cancer stem cell is selected for administration to that mammal. In one embodiment, an ethacrynic derivative of the invention is useful to sensitize solid tumor stem cells, e.g., cancerous pancreatic, liver, colorectal, breast or prostate stem cells, to other anti-cancer therapies, e.g., chemotherapeutics or radiation therapy. In some embodiments, an ethacrynic derivative of the invention may sensitize breast cancer stem cells, for instance, CD24⁺, ESA⁺, CD44⁺, CD133⁺, and/or Sca-1⁺ cells, pancreatic cancer stem cells, e.g., ESA⁺ cells, prostrate cancer stem cells, e.g., CD44⁺, CD49f⁺, CD133⁺, P63⁺ and/or Sca-1⁺ cells, or intestinal cancer stem cells, e.g., NCAM⁺, CD34⁺, Thy-1⁺, c-Kit⁺ and/or Flt-3⁺cells, to other anti-cancer therapies.

In one embodiment, the invention provides a method to prevent, inhibit or treat a disease or disorder associated with NF-kB, e.g., aberrant NF-kB expression or activity, in a mammal in need of such therapy. The method includes administering an effective amount of at least one compound of the invention to the mammal, e.g., a human. In one embodiment, the at least one compound is intravenously administered. In one embodiment, the compound is orally administered, e.g., in tablet form.

In one embodiment, the invention provides a method to prevent, inhibit or treat inflammatory disorders associated with NF-kB in a mammal in need of such therapy. The method includes administering an effective amount of at least one compound of the invention to the mammal, e.g., a human. In one embodiment, the at least one compound is intravenously administered. In one embodiment, the compound is orally administered, e.g., in tablet form.

The invention also provides a pharmaceutical composition comprising one or more of the compounds described herein, or a pharmaceutically acceptable salt thereof, in combination with a pharmaceutically acceptable diluent or carrier. Further, the invention provides a pharmaceutical composition comprising at least one of the compounds disclosed herein in combination with other known anti-cancer compounds.

Thus, the invention provides compounds for use in medical therapy, such as agents that alter Wnt signaling, inhibit the growth or survival of tumor cells, e.g., tumor cells that overexpress Wnt signaling genes, or prevent, inhibit or treat disorders or diseases associated with NF-kB, for instance, prevent, inhibit or treat inflammatory disorders associated with NF-kB, optionally in conjunction with other compounds. Accordingly, the compounds of the invention are useful to inhibit or treat cancer, e.g., leukemia, lymphoma, malignant gliomas, prostate cancer, ovarian cancer, colon cancer, breast cancer, neuroblastoma, lung cancer, or other proliferative diseases. Also provided is the use of the compounds for the manufacture of a medicament to inhibit tumor cell growth or survival, inhibit or treat cancer, or inhibit metastases.

### BRIEF DESCRIPTION OF THE DRAWINGS

FIG. 1. Inhibition of Wnt/β-catenin signaling by EA. (A) HEK293 cells carrying a Wnt responsive reporter (Super 8XTOPflash) were treated with Wnt3a (10 nM) and increasing concentrations of EA (20, 25, 30, 35 μM) for 18 hours. Cells were lysed and luciferase activity was quantified. Total protein levels were determined by Bradford Assay and serve as a control for total cell number. (B) HEK293 cells were co-transfected with TOPflash reporter construct, along with expression plasmids for Wnt1, Wnt3, LRP6, Dvl and β-catenin as indicated. After transfection for 24 hours, the cells were treated with 50 μM EA for another 24 hours, and then luciferase activities were determined. (C) HEK293 cells were transfected with FOPflash reporter with or without an expression plasmid for β-catenin. After transfection, the cells were treated with 50 μM EA for another 24 hours. (D) HEK293 cells were transfected with NFAT reporter and expression plasmid for NFATc. The cells were treated with 50 μM EA for 24 hours, and then harvested, and extracted for determination of luciferase activities. The results are expressed as fold induction of luciferase activity compared to the basal level, and are the means of three experiments±SEM.

FIGS. 2A-D. Effect of selected EA amides on CLL cell viability in vitro.

FIG. 3. Wnt/beta-catenin pathway assays.

FIG. 4. Selective cytotoxicity of EA to CLL cells. Primary CLL cells or normal peripheral blood mononuclear cells (PBMC) were treated with increasing concentrations of EA for 48 hours. The cell viability was measured by MTT assay. The control condition was a 2-day incubation of the cells in the medium alone, and the viability expressed as the percentage with respect to this control.

FIG. 5. Effect of EA on LEF-1, cyclin D1, fibronectin and Fzd5 expression in CLL cells. CLL cells from three patients were treated with increasing amounts of EA for 16 hours. The mRNA levels of LEF-1, cyclin D1, fibronectin and Fzd5 were compared by real-time PCR. Total RNA input was normalized based on the concentration of 18S RNA.

FIG. 6. EA binds to LEF-1 in primary CLL and SW480 cells. (A) lysates from CLL cells exposed to 10 μM EA for 8 hours or 24 hours were immunoprecipitated with anti-LEF-1 antibody. The immune complexes were analyzed by immunoblotting with anti-LEF-1 and anti-EA antibodies. (B) SW480 cells were treated with indicated amounts of EA for 16 hours. Cell lysates were immunoprecipitated with anti-β-catenin antibody (Santa Cruz Biotechnology, Santa Cruz, Calif.). The immune complexes were analyzed by immunoblotting with anti-EA and anti-β-catenin antibodies. (C) SW480 cells were exposed to 50 μM of EA for 16 hours and cell lysates were immunoprecipitated with anti-β-catenin antibody. The proteins in the immunoprecipitates were resolved by SDS-PAGE, transferred, and probed with indicated antibodies. The LEF-1 protein band, as confirmed by reactivity with LEF-1 specific antibody, stained positive with the anti-EA antibody only in the drug treated samples.

FIG. 7. EA destabilizes the LEF-1/β-catenin complex. (A) SW480 cells were treated with increasing amounts of EA for 16 hours. Cells were lysed, and IP was completed with anti-β-catenin monoclonal antibody. The immune complexes were analyzed by immunoblotting with anti-LEF-1, anti-α-catenin and anti-β-catenin antibodies. (B) EA inhibits Wnt/β-catenin signaling in SW480 cells. SW480 cells were transfected with TOPflash reporter and control plasmid pCMXβgal. After transfection for 24 hours, cells were treated with increasing concentrations of EA for another 24 hours as indicated. Cells were then harvested and luciferase values were determined. The results are expressed as relative luciferase activity (%) normalized to a β-galactosidase control.

FIG. 8. N-acetyl-L-cysteine (NAC) prevents EA-mediated effects on the Wnt/β-catenin pathway and on CLL survival. (A) prevention of EA-mediated inhibition of Wnt/β-catenin signaling by free thiols. HEK293 cells were co-transfected with TOPflash reporter vector, and with a Dvl vector to activate signaling. The transfected cells were treated with 50 μM EA, 1 mM NAC, 100 μM PDTC, or 100 μM BHA, as indicated in the figure. After 24 hours incubation, cell extracts were assayed for luciferase activities. (B) rescue of CLL cells from EA-induced apoptosis by NAC. Primary CLL cells were treated with 3 μM EA, 1 mM NAC, 100 μM BHA or combined treatment as indicated. After treatment for 48 hours, the cells were stained with DiOC₆ and PI and analyzed by flow cytometry. Note that NAC, but not other anti-oxidants, protected the CLL cells from EA-induced apoptosis.

### DETAILED DESCRIPTION OF THE INVENTION

### Definitions

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the methods of the present invention. Such organisms include, but are not limited to, mammals such as humans. In the context of the invention, the term “subject” generally refers to an individual who will receive or who has received treatment (e.g., administration of a compound of the invention, and optionally one or more anticancer agents) for cancer.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)O—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl, (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

The alkenyl can optionally be substituted with one or more alkyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃), 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylidenyl can optionally be interrupted with one or more non-peroxide oxy (—O—), thio (—S), carbonyl (—C(═O)—), carboxy (—C(═O)O—), sulfonyl (SO) or sulfoxide (SO₂).

“Alkenylidenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Exemplary alkoxy groups include, e.g., methoxy, ethoxy, n-propoxy, iso-propoxy, n-butoxy, Pert-butoxy, sec-butoxy, n-pentoxy, n-hexoxy, 1,2-dimethylbutoxy, and the like.

The alkoxy can optionally be substituted with one or more alkyl, alkylidenyl, alkenylidenyl, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Exemplary aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂-)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH. The term “nitro” refers to -NO₂. The term “trifluoromethyl” refers to —CF₃. The term “trifluoromethoxy” refers to —OCF₃. The term “cyano” refers to —CN. The term “hydroxy” or “hydroxyl” refers to —OH. The term “oxy” refers to —O—. The term “thio” refers to —S—. The term “thioxo” refers to (═S). The term “keto” refers to (═O).

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein a carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

The term “protecting group” refers to any group that, when bound to a hydroxyl, nitrogen, or other heteroatom, prevents undesired reactions from occurring at the sight of the heteroatom, and which group can be removed by conventional chemical or enzymatic steps to reestablish the ‘unprotected’ hydroxyl, nitrogen, or other heteroatom group. When an amine used to form an amide of ethacrynic acid includes a group that can react with ethacrynic acid chloride besides the intended amine group, a protecting group can be used to protect the reactive group prior to forming the amide. The use of protecting groups is well known to those of skill in the art. Certain removable protecting groups include conventional substituents such as, for example, allyl, benzyl, acetyl, chloroacetyl, thiobenzyl, benzylidine, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a heteroatom functionality and later selectively removed either by chemical or enzymatic methods in conditions compatible with the nature of the reaction and product.

A large number of protecting groups and corresponding chemical cleavage reactions are described in Protective Groups in Organic Synthesis, Theodora W. Greene (John Wiley & Sons, Inc., New York, 1991), which is incorporated herein by reference. Greene describes many nitrogen protecting groups, for example, amide-forming groups. In particular, see Chapter 1, Protecting Groups: An Overview, pages 1-20; Chapter 2, Hydroxyl Protecting Groups, pages 21-94; Chapter 4, Carboxyl Protecting Groups, pages 118-154; and Chapter 5, Carbonyl Protecting Groups, pages 155-184. See also Kocienski, Philip J.; Protecting Groups (Georg Thieme Verlag Stuttgart, New York, 1994), which is also incorporated herein by reference.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

The term “biochemical modulating agent” is an agent given as an adjunct to anti-cancer therapy, which serves to potentate its antineoplastic activity, as well as counteract the side effects of the active agent, e.g., an antimetabolite.

Obviously, numerous modifications and variations of the present invention are possible in light of the teachings herein. It is therefore to be understood that within the scope of the appended claims, the invention may be practiced otherwise than as specifically described herein.

### Uses of Derivatives of the Invention

The ethacrynic compounds of the present invention are useful in medical therapy. In one embodiment, the compounds of the present invention are useful in to alter Wnt signaling, e.g., inhibit Wnt signaling, prevent, inhibit or treat inflammatory disorders, e.g., asthma, allergies, autoimmune disorders, and delayed type hypersensitivity, inhibit or treat cancer and/or to sensitize cancer stem cells to anti-cancer treatments ex vivo or in vivo. As such, in one embodiment, the compounds of the present invention are useful in treating cancer in mammals (e.g., humans), as well inhibiting tumor cell growth in mammals. The cancer may be a leukemia, lymphoma or a solid tumor, e.g., one originating from or located in the ovary, breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, or the heart. In one embodiment, the ethacrynic derivatives of the invention are employed to inhibit or treat leukemia. In one embodiment, the ethacrynic derivatives of the invention are employed to inhibit or treat lymphoma. In one embodiment, the ethacrynic derivatives of the invention are employed to inhibit or treat a hematopoietic cancer such as CLL. Additionally, the compounds of the present invention can be administered locally or systemically, alone or in combination with one or more anti-cancer agents. In one embodiment, the ethacrynic derivatives of the invention are orally administered. In one embodiment, the ethacrynic derivatives of the invention are intravenously administered.

### Combination Therapies

The ethacrynic derivatives of the invention may be administered in combination with other active agents including a chemotherapeutic agent. For example, in one embodiment, the ethacrynic derivatives of the invention are administered in conjunction (sequentially or concurrently) with a taxane, e.g., docetaxel or paclitaxel. Paclitaxel may be administered on a weekly schedule, at doses 60-100 mg/m² administered over 1 hour, weekly, or 2-3 weekly doses followed by a one week rest. In one embodiment, paclitaxel is administered intravenously over 3 hours at a dose of 175 mg/m² over 24 hours at a dose of 135 mg/m². In patients previously treated with therapy for carcinoma, paclitaxel can be injected at several doses and schedules. In one embodiment, paclitaxel is administered intravenously at 135 mg/m² or 175 mg/m² over 3 hours every 3 weeks. These doses may be altered as needed or desired.

In one embodiment, the ethacrynic derivatives of the invention are administered in conjunction (sequentially or concurrently) with an alkylating agent; hormonal agent (e.g., estramustine, tamoxifen, toremifene, anastrozole, or letrozole); antibiotics (e.g., plicamycin, bleomycin, mitoxantrone, idarubicin, dactinomycin, mitomycin, or daunorubicin); antimitotic agent (e.g., vinblastine, vincristine, teniposide, or vinorelbine, available as Navelbine); topoisomerase inhibitor (e.g., topotecan, irinotecan, etoposide, or doxorubicin, e.g., CAELYX or Doxil, pegylated liposomal doxorubicin hydrochloride); or other agent (e.g., hydroxyurea, altretamine, rituximab, L-asparaginase, or gemtuzumab ozogamicin); or a biochemical modulating agent, e.g., imatib, EGFR inhibitors such as EKB-569 or other multi-kinase inhibitors, e.g., those that target serine/threonine and receptor tyrosine kinases in both the tumor cell and tumor vasculature, or immunomodulators (e.g., interferons, IL-2, or BCG). Examples of suitable interferons include interferon-alpha, interferon-beta, interferon-gamma, and mixtures thereof.

In one embodiment, the ethacrynic derivatives of the invention are administered in conjunction (sequentially or concurrently) with an antineoplastic alkylating agent, e.g., those described in U.S. Publication No. 20020198137A1. Antineoplastic alkylating agents may be classified according to their structure or reactive moiety, into several categories which include nitrogen mustards, such as meclorethamine, cyclophosphamide, ifosfamide, melphalan, and chlorambucil; azidines and epoxides, such as thiotepa, mitomycin C, dianhydrogalactitol, and dibromodulcitol; alkyl sulfinates, such as busulfan; nitrosoureas, such as bischloroethylnitrosourea, cyclohexyl-chloroethylnitrosourea, and methylcyclohexylchloroethylnitrosourea; hydrazine and triazine derivatives, such as procarbazine, dacarbazine, and temozolomide; streptazoin, melphalan, chlorambucil, carmustine, methclorethamine, lomustine, and platinum compounds. Platinum compounds are platinum containing agents that react preferentially at the N7 position of guanine and adenine residues to form a variety of monofunctional and bifunctional adducts. These compounds include cisplatin, carboplatin, platinum IV compounds, and multinuclear platinum complexes.

The following are representative examples of alkylating agents and possible routes of administration: meclorethamine is commercially available as an injectable; cyclophosphamide is commercially available as an injectable and in oral tablets; ifosfamide is commercially available as an injectable; melphalan is commercially available as an injectable and in oral tablets; chlorambucil is commercially available in oral tablets; thiotepa is commercially available as an injectable; mitomycin is commercially available as an injectable; busulfan is commercially available as an injectable and in oral tablets; lomustine is commercially available in oral capsules; carmustine is commercially available as an intracranial implant and as an injectable; procarbazine is commercially available in oral capsules; temozolomide is commercially available in oral capsules; cisplatin is commercially available as an injectable; carboplatin is commercially available as an injectable; and oxiplatin is also commercially available.

In one embodiment, the ethacrynic derivatives of the invention are administered in conjunction (sequentially or concurrently) with an antineoplastic antimetabolite, such as is described in U.S. Publication No. US 20050187184 or 20020183239. An “antimetabolite” means a substance which is structurally similar to a critical natural intermediate (metabolite) in a biochemical pathway leading to DNA or RNA synthesis which is used by the host in that pathway, but acts to inhibit the completion of that pathway (i.e., synthesis of DNA or RNA). More specifically, antimetabolites typically function by (1) competing with metabolites for the catalytic or regulatory site of a key enzyme in DNA or RNA synthesis, or (2) substitute for a metabolite that is normally incorporated into DNA or RNA, and thereby producing a DNA or RNA that cannot support replication. Major categories of antimetabolites include (1) folic acid analogs, which are inhibitors of dihydrofolate reductase (DHFR); (2) purine analogs, which mimic the natural purines (adenine or guanine) but are structurally different so they competitively or irreversibly inhibit nuclear processing of DNA or RNA; and (3) pyrimidine analogs, which mimic the natural pyrimidines (cytosine, thymidine, and uracil), but are structurally different so thy competitively or irreversibly inhibit nuclear processing of DNA or RNA. Exemplary antimetabolites include but are not limited to 5-Fluorouracil (e.g., a topical cream, a topical solution or as an injectable); floxuradine (2′-deoxy-5-fluorouridine); thioguanine (2-amino-1,7-dihydro-6-H-purine-6-thione); cytarabine (4-amino-1-(beta)-D-arabinofuranosyl-2(1H)-pyrimidinone, e.g., in a liposomal injectable or a liquid injectable; fludarabine (9-H-Purin-6-amine, 2-fluoro-9-(5-O-phosphono-(beta)-D-α-rabinofuranosyl); 6-Mercaptopurine (1,7-dihydro-6H-purine-6-thione); methotrexate (MTX; N44-[[(2,4-diamino-6-pteridinyl)methyl]methylamino]benzoyl]-L-glutamic acid) (e.g., a liquid injectable or oral tablets); gemcitabine (2′-deoxy-2′,2′-difluorocytidine monohydrochloride ((beta)-isomer)); capecitabine (5′-deoxy-5-fluoro-N-[(pentyloxy)carbonyl]-cytidine); pentostatin ((R)-3-(2-deoxy-(beta)-D-erythro-pentofuranosyl)-3,6,7,-8-tetrahydroimidazo[4,5-d][1,3]diazepin-8-ol)yl; trimetrexate (2,4-diamino-5-methyl-6-[(3,4,5-trimethoxyanilino)methyl]quinazoline mono-D-glucuronate); and cladribine (2-chloro-6-amino-9-(2-deoxy-(beta)-D-erythropento-furanosyl)purine).

In one embodiment, the ethacrynic derivatives of the invention are administered in conjunction (sequentially or concurrently) with a kinase inhibitor such as a multi-kinase inhibitor that targets serine/threonine and receptor tyrosine kinases in both the tumor cell and tumor vasculature. Examples of suitable kinase inhibitors are Sorafenib, Zarnestra (R115777, tipifarnib), suntinib, avastin, ISIS 5132, and MEK inhibitors such as CI-1040 or PD 0325901.

### Pharmaceutical Compositions and Routes of Administration

The compounds of the invention can be formulated as pharmaceutical compositions and administered to a mammalian host, such as a human patient, in a variety of forms adapted to the chosen route of administration, e.g., orally or parenterally, by intravenous, intramuscular, topical or subcutaneous routes.

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, exemplary methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for instance, in the range of 6 to 90 mg/kg/day, such as in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, e.g., about 1 to 50 μM or about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to alter Wnt signaling, inhibit tumor cell proliferation, survival or metastases, inhibit cancer stem cell proliferation or survival, or prevent inhibit or treat disorders or diseases associated with associated with NF-kB, such as inflammatory disorders or diseases, may be determined in vitro or using pharmacological models which are well known to the art, or using the procedures described below.

### Exemplary Compounds

In one embodiment, a compound of the invention has formula (I):

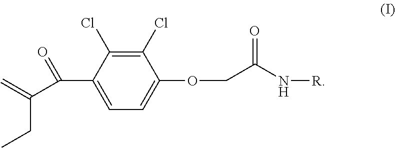

In one embodiment, R is an alkyl nitrate, for example, a (C₁-C₆)alkyl nitrate, such as nitromethyl.

In one embodiment, R is a substituted aryl, substituted phenyl, or hydroxyphenyl.

In one embodiment, R is a substituted aryl, substituted phenyl, or carboxamidophenyl.

In one embodiment, R is hydroxyl.

In one embodiment, R is a substituted aryl, substituted phenyl, or carboxyphenyl.

In one embodiment, R is an alkyl, optionally substituted with thiol and/or alkyl carboxylate.

In one embodiment, R is a substituted aryl, substituted phenyl, or cyanophenyl.

In one embodiment, R is a substituted aryl, substituted phenyl, or phenyl optionally substituted with halo and/or carboxy.

In one embodiment, R is a substituted aryl, substituted phenyl, or phenyl optionally substituted with one to five groups selected from carboxy or alkoxy.

In one embodiment, R is a substituted aryl, substituted phenyl, or phthalimido.

In one embodiment, R is a heteroaryl or benzothiazole.

In one embodiment, R is a substituted alkyl, e.g., N-morpholinoalkyl.

In one embodiment, R is a substituted aryl, e.g., a substituted phenyl, such as phenyl substituted with 2-ethanoic acid.

In one embodiment, R is an alkyl optionally substituted with carboxy, 2-ethanoic acid.

In one embodiment, R is optionally a substituted cycloalkylalkyl, carboxycyclohexylalkyl.

In one embodiment, R is optionally a substituted heterocycle, piperidine optionally substituted with alkyl acetate.

In one embodiment, R is an alkyl substituted with indole, such as indolylalkyl.

In one embodiment, R is a substituted heteroaryl, e.g., a substituted pyridine, such as carboxypyridine.

In one embodiment, R is a substituted aryl, such as a substituted phenyl, e.g., phenyl substituted with hydroxy and/or carboxy.

In one embodiment, R is a substituted aryl, for instance, a substituted phenyl, such as phenyl substituted with alkyl carboxylate.

In one embodiment, R is a substituted aryl, e.g., a substituted phenyl, for instance 1,4-dihydrophthalazine-1,4-diol.

In one embodiment, R is a substituted aryl, including a substituted phenyl, for instance, 4-methyl-2H-chromen-2-one.

In one embodiment, R is a substituted aryl, including a substituted phenyl, e.g., (S)-3-ethyl-3-phenylpiperidine-2,6-dione.

In one embodiment, R is a substituted aryl, e.g., a substituted phenyl, including phenyl substituted with optionally substituted benzyl, wherein the substitution can be a heterocycle.

In one embodiment, R is a substituted aryl, e.g., a substituted phenyl, such as acetylphenyl.

In one embodiment, R is a substituted aryl, for instance, a substituted phenyl, or heterocycle, e.g., 1H-benzo[d]imidazole.

In one embodiment, R is a substituted heterocycle, such as a substituted thiazole.

In one embodiment, R is a substituted aryl, including a substituted phenyl, e.g., diethyl 2-benzamidopentanedioate.

In one embodiment, R is a substituted aryl, including anthracene-9,10-dione.

In one embodiment, R is a substituted alkyl, such as a heteroaryl substituted alkyl, e.g., 1-propylpyrrolidin-2-one.

In one embodiment, R is a substituted aryl, including a substituted phenyl, e.g., phenyl substituted with hydroxy and/or nitro, phenyl substituted with halo and/or hydroxyl, phenylsulfonic acid, alkyl cinnamate, cinnamic acid or N-hydroxycinnamamide.

In one embodiment, R is a substituted aryl, such as a substituted naphthyl, e.g., carboxynaphthyl.

In one embodiment, R is a hydroxyalkyl, for example, a hydroxy(C₁-C₆)alkyl, such as hydroxyethyl.

In one embodiment, a compound of the invention includes a protecting group (e.g. acetyl, benzyl, benzyloxy, benzyloxycarbonyl, (C₁-C₆)alkyl, phenyl or benzyl ester amide, or a-methylbenzyl amide). Other suitable protecting groups are known to those skilled in the art (See for example, Greene, T. W.; Wutz, P. G. M. Protecting Groups In Organic Synthesis, 2^{nd} edition, John Wiley & Sons, Inc., New York (1991) and references cited therein).

TABLE 1
Exemplary Ethacrynic Amides

| Entry | ID | R |
|---|---|---|
| 1 | 1×184-2 | 2-hydrxyethyl |
| 2 | 1×184-3 | 2-nitrooxyethyl |
| 3 | 1×202-2 | 4-hydroxy-phenyl |
| 4 | 1×203-3 | 4-carbamoyl-phenyl |
| 5 | 1×203-2 | hydroxyl |
| 6 | 1×202-1 | 4-carboxypheneyl |
| 7 | 1×204 | 1-ethoxycarbonyl-2- |
| | | mercapto-ethyl |
| 8 | 1×204-2 | 4-cyanophenyl |
| 9 | 1×205-1 | 3-carboxy-4-chlorophenyl |
| 10 | 1×205-2 | 2-carboxy-5-chlorophenyl |
| 11 | 1×205-3 | 2-carboxy-4,5-dimethoxy- |
| | | phenyl |
| 12 | 1×214 | 1,3-dioxo-2,3-dihydro-1H- |
| | | isoindolyl |
| 13 | 1×215 | benzothiazol-2-yl |
| 14 | 1×220 | 2-morpholin-4-yl-ethyl |
| 15 | 1×222 | 4-carboxymethylphenyl |
| 16 | 1×223 | Carboxymethyl |
| 17 | 1×224 | 4-carboxy-cyclohexyl |
| | | methyl |
| 18 | 1×225 | 1-methoxycarbonyl- |
| | | methyl-piperidin-4-yl |
| 19 | 1×234-2 | 3-carboxyphenyl |
| 20 | 1×234-3 | 2-(1H-indol-3-yl)-ethyl |
| 21 | 1×237-2 | 3-carboxy-6-pyridinyl |
| 22 | 1×238 | 3-hydroxy-4-carboxy-phenyl |
| 23 | 1×240 | 4-methoxycarbonyl-phenyl |
| 24 | 1×241 | 1,4-dihydroxy-1,4-dihydro- |
| | | phthalazin-6-yl |
| 25 | 1×242 | 4-methyl-2-oxo-2H-chromen- |
| | | 7-yl |
| 26 | 1×243 | 4-(3-ethyl-2,6-dioxo-piperidin- |
| | | 3-yl)-phenyl |
| 27 | 1×244-1 | 4-[4-(3,5-dioxo-4-aza- |
| | | tricyclo[5.2.1.02,6]dec-8-en-4- |
| | | yl)-benzyl]-phenyl |
| 28 | 1×244-2 | 4-acetyl-phenyl |
| 29 | 1×244-3 | 3H-benzoimidazol-5-yl |
| 30 | 1×245-3 | 4-(Carboxy-methoxyimino- |
| | | methyl)-thiazol-2-yl |
| 31 | 1×245-2 | 4-(1,3-bis-ethoxycarbonyl- |
| | | propylcarbamoyl)-phenyl |
| 32 | 1×246-2 | 9,10-dioxo-9,10-dihydro- |
| | | anthracen-2-yl |
| 33 | 1×246-1 | 3-(2-oxo-pyrrolidin-1-yl)- |
| | | propyl |
| 34 | 1×236-2 | 4-hydroxy-3-nitro-phenyl |
| 35 | 1×236-1 | 3-chloro-4-hydroxy-phenyl |
| 36 | 1×235 | 4-sulfo-phenyl |
| 37 | 1×237-1 | 2-carboxy-6-naphthalenyl |
| | | methyl |
| 38 | 1×251 | 4-(2-Ethoxycarbonyl)-vinyl)- |
| | | phenyl |
| 39 | 1×189-2 | Ethyl ester of EA |
| 40 | EA | |
| | (ethacrynic acid) | |

The invention will be described by the following non limiting examples.

### EXAMPLE I

### Amide Derivatives of Ethacrvnic Acid: Synthesis and Evaluation as Antagonists of Wnt/β-catenin Signaling and CLL Cell Survival

### Materials and Methods

### Wnt Signaling Inhibition

To determine the specificity of compounds on Wnt/□-catenin pathway inhibition, CellSensor LEF/TCF-bla SW480 cell-based assay (Invitrogen, Carlsbad, Calif.) was used according to the supplier's instructions, but modified for a 96 well format. Cells were plated at 25,000 cells/well in assay medium in 96-well black plates with clear bottom (Corning) the day prior to compound treatment. Compounds were added to cells at a final concentration ranging from 33.3 μM to 0.5 μM, incubated for 20 hours and then combined with LiveBLAzer™-FRET B/G Substrate (CCF4-AM) for 2 hours at room temperature. Fluorescence emission values at 465 nm and 535 nm were obtained using a standard fluorescence plate reader and the 465/535 ratios were calculated for each treatment (n=2 for each data point). Results were normalized to untreated control cells (set at 100%, n=4), plotted as % of control, and EC₅₀ determined using Prism 4.0a software (GraphPad).

### Human Samples

Blood samples were collected by the Chronic Lymphocytic Leukemia Research Consortium, after obtaining informed consent from patients fulfilling diagnostic criteria for CLL, at all disease stages. Institutional review board approval was obtained from University of California San Diego for the procurement of patient samples in this study, in accordance with the Declaration of Helsinki. The patients in this study have given written informed consent to publication of their case details.

### Chemical Reagents

Ethacrynic acid (EA), N-acetyl-L-cysteine (NAC), pyrrolidinedithiocarbamate ammonium salt (PDTC), and 3-t-butyl-4-hydroxyanisole (BHA) were from Sigma-Aldrich (St. Louis, Mo.). A Genplus collection of 960 known drugs was obtained from Microsource (Gaylordsville, Conn.).

### Transfection and Screening of Drug Library

The human embryonic kidney cell line HEK293 (American Type Culture Collection, Rockville, Md.) was transfected using the FuGene transfection reagent (Roche Diagnostics GmbH, Mannheim, Germany) according to the manufacturer's instruction.

The reporter plasmids TOPflash and FOPflash were gifts from H. Clevers (University of Utrecht, Utrecht, The Netherlands). The pNFAT-Luc reporter was purchased from BD Biosciences. The expression plasmids encoding Wnt1, Wnt3, LRP6, Dvl, β-catenin and NFATc have been described previously (Lu et al., 2004; Lu et al., 2005).

For screening of the drug library, HEK293 cells were grown for at least 24 hours in 10 cm plates prior to transfection. At 50% confluence, cells were transfected with 5 μg of TOPflash reporter, 1 μg expression vector for Dvl, 1 μg of control plasmid pCMXβgal and carrier DNA pcDNA3 plasmid for a total of 10 μg/plate. After transfection for 24 hours, cells were harvested and dispersed in 96-well microtiter plates. Then the cells were treated with the different agents, generally at 10 μM and 50 μM for the initial screen. After overnight incubation, the cells were lysed in 1× potassium phosphate buffer, pH 7.8, containing 1% Triton X-100, and luciferase activities were assayed in the presence of substrate using a microtiter plate luminometer (MicroBeta TriLux, Gaithersburg, Md.). The luciferase values were normalized for variations in transfection efficiency using the β-galactosidase internal control. EA, and other compounds that were scored positive, had ≧30% inhibition of TOPflash activity when compared to the designated control cultures. In other experiments, transient transfections were performed in 12-well plates. HEK293 or SW480 cells were transfected with 0.5 μg of reporter plasmid, 0.1 μg of control plasmid pCMXβgal, 0.1-0.2 μg expression plasmids, and carrier DNA pcDNA3 plasmid for a total of 1 μg/well. After 16 hours, the cells were washed and treated with 50 μM EA or solvent (DMSO) for 24 hours. Then luciferase values were determined. In the Results section, data are expressed as fold stimulation of luciferase activity compared to the basal level. All the transfection results represent means of a minimum of three independent transfections assayed in duplicate, ± the standard error of the mean (SEM).

### Activation of TOPflash Reporter Using Wnt3a

The Super8XTOPflash construct (kindly provided by Dr. R. Moon) was stably transfected into HEK293 cells, and single cell clones were isolated. The stable Super8XTOPflash reporter cell line displays low basal luciferase activity and strong luciferase induction in response to Wnt3a stimulation. Preparation of Wnt3a and Wnt3a stimulations were performed as described in Willert et al. (2003) and Willert et al. (2008).

Cell Viability Assay with 3-[4,5-Dimethylthiazol-2-yl]-2,5-diphenyl Tetrazolium Bromide (MTT)

Primary CLL cells were collected from patients' peripheral bloods after informed consent, and isolated by Ficoll/Hypaque density-gradient centrifugation as previously described in Lu et al. (2004). Normal peripheral blood mononuclear cells were also purified as described in Lu et al. (2004). The cells were resuspended in RPMI 1640 medium with 10% fetal bovine serum (Gemini Bio-Products, West Sacramento, Calif.), and antibiotics at 37° C., 5% CO₂. Cell viability after drug exposure was assessed by MTT assay. Fresh CLL or peripheral blood mononuclear cells were plated at 2.5×10⁵ per well in 96-well plates. After 48 hours, 1/10 volume of 5 mg/ml MTT was added, and cells were incubated at 37° C. overnight. Finally, ½ volume of Lysis buffer was added to the cultures, and ODs at 570 nm were read and recorded.

### General Procedure for EA Derivative Synthesis

To a mixture of 1 mmol of ethacrynic acid in 10 mL of benzene, 1 mL of thionyl chloride was added. The mixture was heated at reflux for 1.5 hours, and solvent was removed in vacuo. Another 10 mL of benzene was added and distilled off again. The residue was dissolved in a small volume of benzene for the next step. The resulting ethacrynic chloride solution was added dropwise to a solution of 1 mmol of amine in pyridine (10 mL) at 0° C. with stirring. The reaction was stirred at ambient temperature for 3 hours, the solvent was distilled off in vacuo, the residue was dissolved in ethyl acetate, and washed with water and brine. The organic layer was dried over anhydrous MgSO₄, and the residue was purified by silica gel column chromatography (dichloromethane:methanol from 100:0 to 100:5) to obtain the pure EA amides shown in Table 1.

Selected data for compound 4: ¹H-NMR (400 MHz, CDCl₃) δ 9.23.(br. 1H), 7.89 (d, J=8 Hz, 1H), 7.70 (d, J=8 Hz, 1H), 7.21 (d, J=8 Hz, 2H), 6.98 (d, J=8 Hz, 2H), 6.20 (br., 1H), 5.98 (d, J=8 Hz, 1H), 5.62 (d, J=12 Hz, 1H), 4.79 (d, J=12 Hz, 1H), 2.77 (s, 2H), 2.44 (q, J=8 Hz, 2H), 1.17 (t, J=8 Hz, 3H). MS (ESI) m/z: 422, [M+H]⁺.

Selected data for compound 6: ¹H-NMR (400 MHz, CDCl₃) δ 9.47.(br. 1H), 8.03 (d, J=8 Hz, 2H), 7.73 (d, J=8 Hz, 2H), 7.21 (d, J=8 Hz, 1H), 6.99 (d, J=8 Hz, 1H), 5.98 (d, J=8 Hz, 1H), 5.62 (d, J=8 Hz, 1H), 4.81 (s, 2H), 2.90 (br., 1H), 2.45 (q, J=8 Hz, 2H), 1.17 (t, J=8, 3H). MS (ESI) m/z: 423, [M+H]⁺.

Selected data for compound 37: ¹H-NMR (400 MHz, CDCl₃) δ 9.30 (br. 1H), 8.57 (s, 1H), 8.40 (s, 1H), 7.95(d, J=8 Hz, 1H), 7.93 (d, J=8 Hz, 1H), 7.86 (d, J=8 Hz, 1H), 7.62 (d, J=8 Hz, 1H), 7.22 (d, J=8 Hz, 1H), 7.04 (d, J=8 Hz, 1H), 5.99 (s, 1H), 5.63 (s, 1H), 4.83 (s, 2H), 2.60 (br., 1H), 2.48 (q, J=8 Hz, 2H), 1.17 (t, J=8, 3H). MS (ESI) m/z: 473, [M+H]⁺.

Selected data for compound 40: ¹H-NMR (400 MHz, CDCl₃) δ 10.70 (br. 1H), 10.38.(br. 1H). 9.00⁻(br. 1H), 7.64 (d, J=1.6 Hz, 2H), 7.50 (d, J=1.6 Hz, 2H), 7.32 (d, J=8.4 Hz, 1H), 7.15 (d, J=8.4 Hz, 1H), 6.37 (s, 1H), 6.33 (s, 1H), 6.06 (s, 1H), 5.56 (s, 1H), 4.97 (s, 2H), 2.36 (q, J=6.8 Hz, 2H), 1.07(t, J=7.6 Hz, 3H). MS (ESI) m/z: 463, [M+H]⁺.

### Results and Discussion

Recently, it has been demonstrated that the Wnt signaling pathway is activated in CLL cells, and that uncontrolled Wnt/β-catenin signaling may contribute to the defect in apoptosis that characterizes this malignancy (Rosenwald et al. 2001; Lu et al., 2004). Therefore, the Wnt/β-catenin signaling molecules are attractive candidates for developing targeted therapies for CLL.

Cell-based screens of libraries of natural products and synthetic small molecules have provided useful tools for the study of complex cellular processes. Indeed, a number of small molecules have been identified that modulate Wnt/β-catenin signaling, including NSAIDs (Lu et al., 2005), quercetin (Park et al., 2005), ICG-001 (Teo et al. 2005), and others (Barker et al., 2006).

### Inhibition of Wnt/⊕-Catenin Signaling by EA

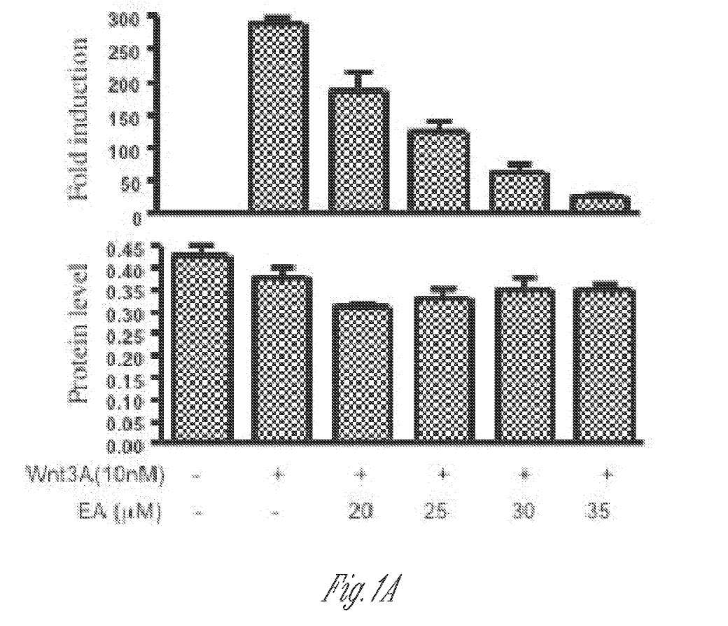

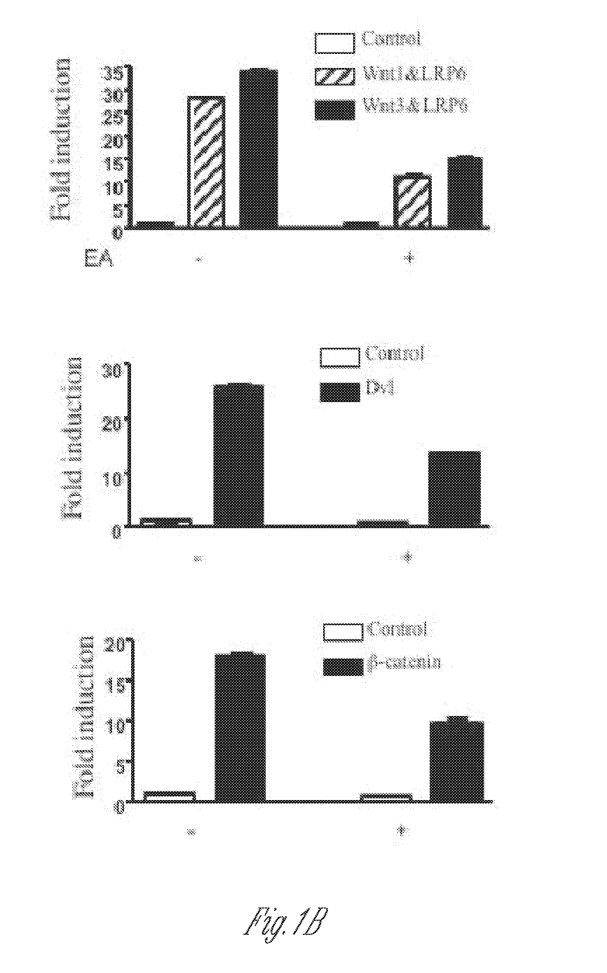

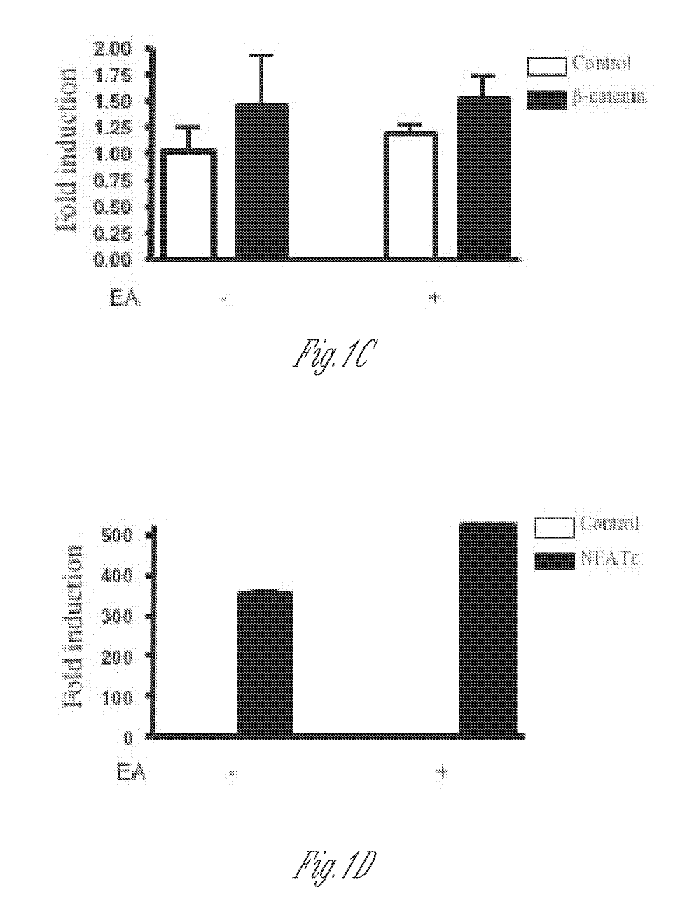

To identify novel antagonists of Wnt/β-catenin signaling, a 96-well plate-based TOPflash reporter system was used to screen the Gen-plus drug library (Microsource) that contains 960 FDA-approved drugs. In this system, transfected Dvl (an upstream activator of the Wnt/β-catenin pathway) stimulated TCF/LEF response elements in the TOPflash reporter gene. In accord with earlier research, the screen identified several non-steroidal anti-inflammatory drugs (NSAIDs), PPARγ, and RXRα ligands as Wnt antagonists (Lu et al., 2005). However, no other compound classes inhibited reporter gene activity, including many known cytotoxic agents. Surprisingly, the screen identified ethacrynic acid (EA), but not other diuretic agents, as a Wnt/β-catenin inhibitor. To further determine the inhibitory effect of EA on Wnt signaling, the stable SuperTOPflash reporter cell line was treated with Wnt3a and increasing concentrations of EA. Wnt3a induced transcriptional activity of the SuperTOPflash reporter 300-fold above the basal levels. EA blocked Wnt3a-induced transcription in a dose-dependent manner (FIG. 1A).

To explore possible targets of EA in the Wnt/β-catenin pathway, the TOPflash reporter was activated by Wnt1/LRP6 or Wnt3/LRP6, Dvl and β-catenin, respectively, in transient transfection assays. Treatment with EA reduced Wnt1/LRP6 or Wnt3/LRP6, Dvl, and β-catenin-induced transcription in HEK293 cells (FIG. 1B). This action was specific, since the drug had no effect on the FOPflash reporter (FIG. 1C). In addition, EA did not block NFATc-mediated transcription from a NFAT reporter (FIG. 1D). These results suggest that EA may specifically inhibit Wnt/β-catenin signaling through targeting either β-catenin itself or its downstream factors.

Ethacrynic acid, a once commonly used loop diuretic drug, was previously shown to be uniquely cytotoxic toward primary CLL cells (Twentyman et al., 1992). However, EA is not ideal as a chemotherapeutic agent for CLL treatment due to its diuretic properties and relative lack of potency. Therefore, some amide derivatives of EA were synthesized and evaluated for inhibition of Wnt signaling and for decreasing the survival of cells from CLL patients.

The preparation of the amide derivatives of EA was accomplished by refluxing EA in benzene with thionyl chloride to form the EA acyl chloride intermediate followed by reaction in pyridine with desired amines.

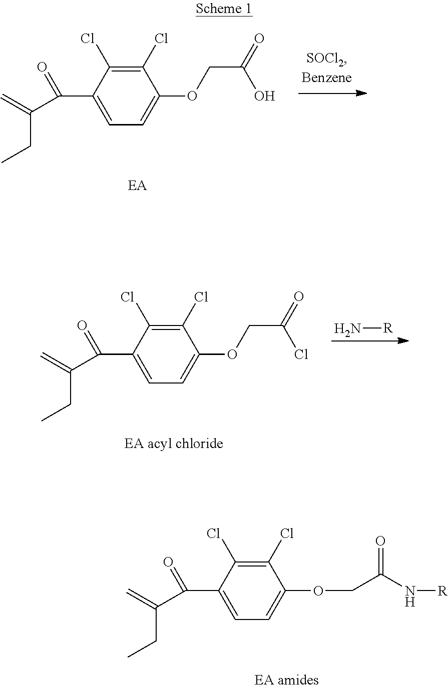

Thus, by this procedure over forty compounds were prepared and evaluated. Furthermore, to explore the contribution of the C—C double bond of the α-β unsaturated carbonyl function to the bioactivity, the double bond of EA was reduced by catalytic hydrogenation (Woltersdorf et al., 1977) to afford EA-R (42). In addition, a few simple alkyl esters of EA and a “truncated” decarboxylated version of EA (compound 43) were prepared. Although EA esters were reported to kill CLL cells at low micromolar concentrations (Zhao et al., 2007), those esters along with 43 were also highly toxic to peripheral blood mononuclear cells (PBMC) in the assays and were not studied further for Wnt and CLL activity (data not shown).

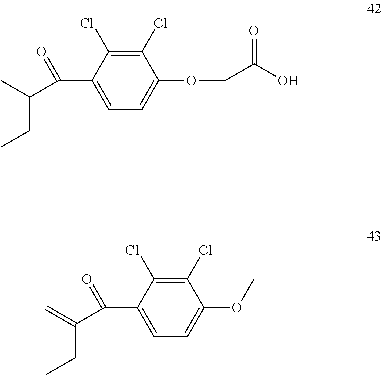

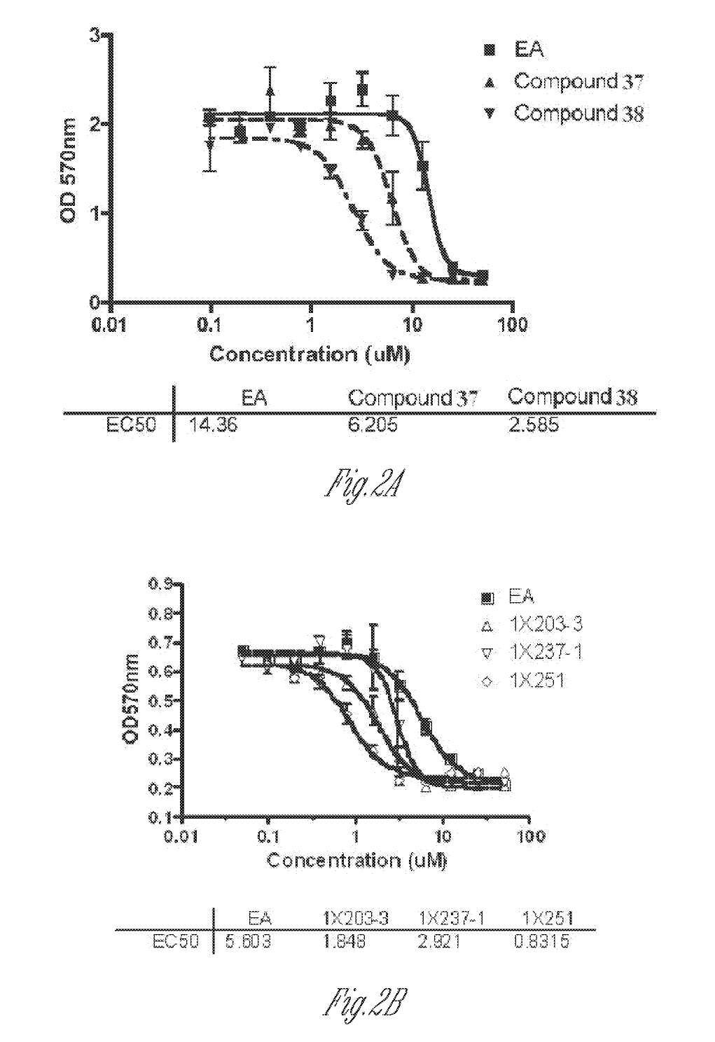

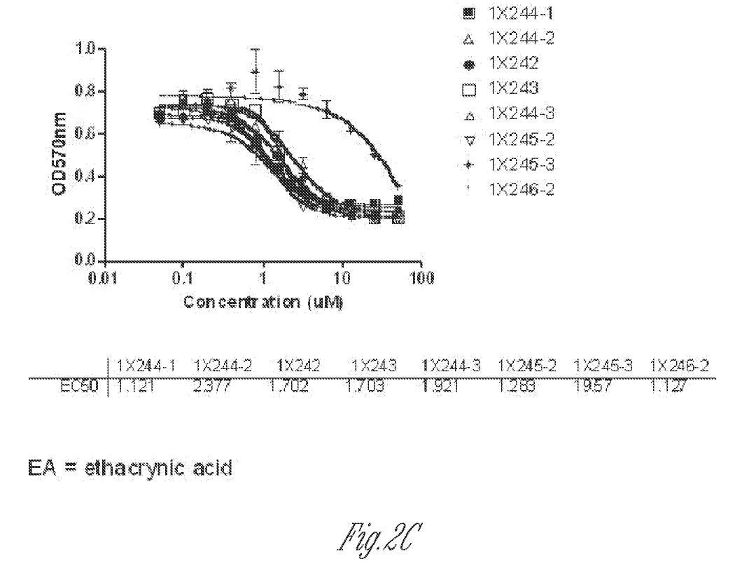

The mechanism of ethacrynic acid cytotoxicity has been attributed to the drug's known capacity to inhibit glutathione S-transferase (GST), causing increased cellular oxidative stress. However, a recent study (Aizawa et al., 2003) showed that the antioxidant N-acetyl-L-cysteine (NAC) protected ethacrynic acid-induced cell death with no effect on cellular glutathione levels, whereas the free radical scavenger 3(2) t-butyl-4-hydroxyanisole (BHA) did not repress ethacrynic acid-induced cell death, suggesting the existence of additional or alternative pathways that are altered by the drug. Since EA is classified as an α,β-unsaturated ketone, its Wnt inhibition activities are most likely due to the alkylation effects on Wnt proteins which are comprised of cysteine-rich glycoproteins (Takahashi et al., 2007). Indeed, inhibition of Wnt signaling by EA can be blocked by adding N-acetyl-L-cysteine or 2-aminoethanethiol to the media prior to testing (see below). Moreover, decreased survival of CLL cells and inhibition of Wnt signaling by EA were completely abrogated after reduction of the α-β double bond by hydrogenation (FIG. 2, EA-R and Table 2, compound 42), suggesting that this Michael acceptor function is essential for its activity.

TABLE 2
Inhibition of Wnt signaling and CLL survival by Ethacrynic amides.

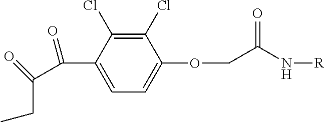

| | | CLL | Wnt | | | CLL | Wnt |
|---|---|---|---|---|---|---|---|
| | | Inhi- | Inhi- | | | Inhi- | Inhi- |
| | | bi- | bi- | | | bi- | bi- |
| | | tion | tion | | | tion | tion |
| En- | | EC₅₀ | IC₅₀ | En- | | EC₅₀ | IC₅₀ |
| try | R | (μM) | (μM) | try | R | (μM) | (μM) |
| 1 | Ethacrynic acid | 9.9 | 32.7 | 21 | 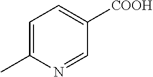 | 14.9 | >50 |
| 2 | 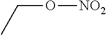 | 4.1 | >50 | 22 | 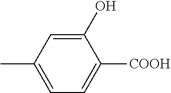 | >25 | >50 |
| 3 | 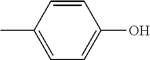 | 6.4 | >50 | 23 | 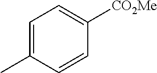 | 4.1 | 11.38 |
| 4 | 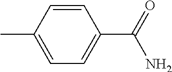 | 3.7 | 4.76 | 24 | 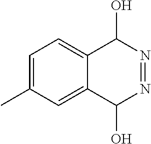 | 8.5 | 6.58 |
| 5 | —OH | 4.5 | 9.89 | 25 | 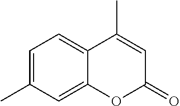 | 2.8 | 2.63 |
| 6 | 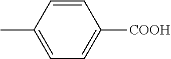 | 5.0 | 5.81 | 26 | 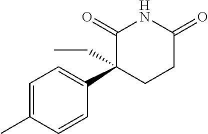 | 2.8 | 4.42 |
| 7 | 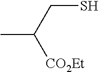 | 4.8 | >50 | 27 | 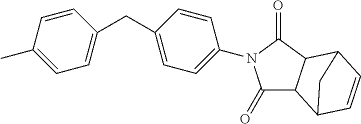 | 1.8 | 2.93 |
| 8 | 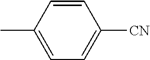 | 7.8 | 6.84 | 28 | 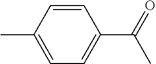 | 3.9 | 5.06 |
| 9 | 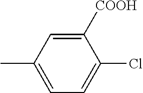 | 15.9 | >50 | 29 | 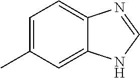 | 3.2 | 3.62 |
| 10 | 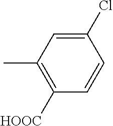 | 13.8 | 9.62 | 30 | 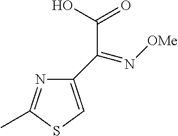 | >25 | >50 |
| | | CLL | Wnt | | | CLL | Wnt |
| | | Inhi- | C. Inhi | | | Inhi- | D. Inhi- |
| | | bi- | bi- | | | bi- | bi- |
| | | tion | tion | | | tion | tion |
| En- | | EC₅₀ | IC₅₀ | En- | | EC₅₀ | IC₅₀ |
| try | R | (μM) | (μM) | try | R | (μM) | (μM) |
| 11 | 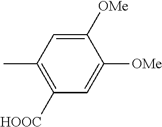 | 21.9 | >50 | 31 | 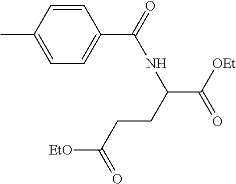 | 2.1 | 2.61 |
| 12 | 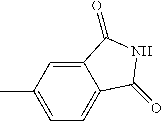 | 2.5 | 4.88 | 32 | 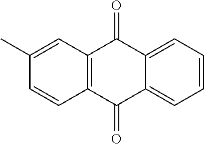 | 2.1 | 2.97 |
| 13 | 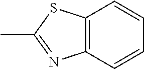 | 1.5 | 4.86 | 33 | 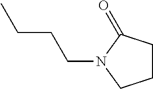 | 13.8 | >50 |
| 14 | 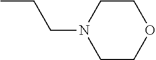 | 6.4 | >50 | 34 | 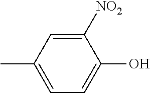 | 7.4 | 4.23 |
| 15 | 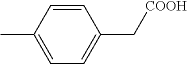 | 3.2 | >50 | 35 | 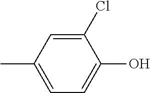 | 5.5 | 3.80 |
| 16 | 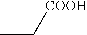 | >25 | >50 | 36 | 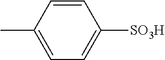 | >25 | >50 |
| 17 | 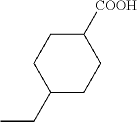 | 8.0 | >50 | 37 | 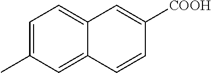 | 3.7 | 2.93 |
| 18 | 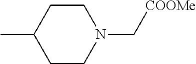 | 10.6 | >50 | 38 | 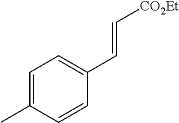 | 1.7 | 3.79 |
| 19 | 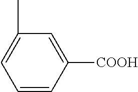 | 20.6 | 5.85 | 39 | 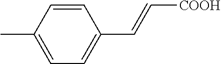 | 3.0 | 2.51 |
| 20 | 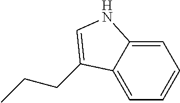 | 5.9 | 10.7 | 40 | 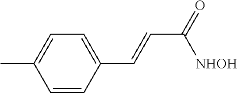 | 2.6 | 1.81 |
| 41 | 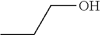 | >25 | >50 | 42 | EA—R | >25 | >50 |

Several compounds were found to effectively decrease CLL survival and antagonize Wnt signaling at low micromolar concentrations (25, 29, 31, 37, 39 and 40). These results correlate with earlier findings that Wnt signaling genes are over-expressed and active in CLL (Lu et al., 2004). It is possible that EA derivatives might inhibit Wnt signaling by covalent modification of sulfhydryl groups of Wnt-dependent genes such as Lef-1 (which is highly expressed in CLL).

Structure-activity trends among the amides in terms of Wnt signaling inhibition revealed that aromatic-containing amides were generally more active than aliphatic amides. Moreover, the larger aromatic substitutions (benzothiazole, phthalimide, naphthyl carboxylic acid, etc.) showed good activity in both systems. It is noteworthy that the IC₅₀s for inhibition of Wnt signaling are consistently lower than the EC₅₀s for inhibition of CLL survival, except for most of the aromatic carboxylic acids. This suggests that the active EA derivatives may have some other target receptor in the cell, a target that may impact CLL survival in addition to the Wnt signaling alone. An example of a possible off-target receptor for the EA derivatives might be inhibition of NF-κB activity through direct inhibition of IKK-β, wherein the cysteine 179 in the activation loop of IKK-β can be covalently modified by Michael acceptors (Rossi et al., 2000). Two well known examples of this are prostaglandin J2 and prostaglandin A1, both of which contain the αβ-unsaturated carbonyl function, and thus the EA derivatives may be acting in a similar manner.

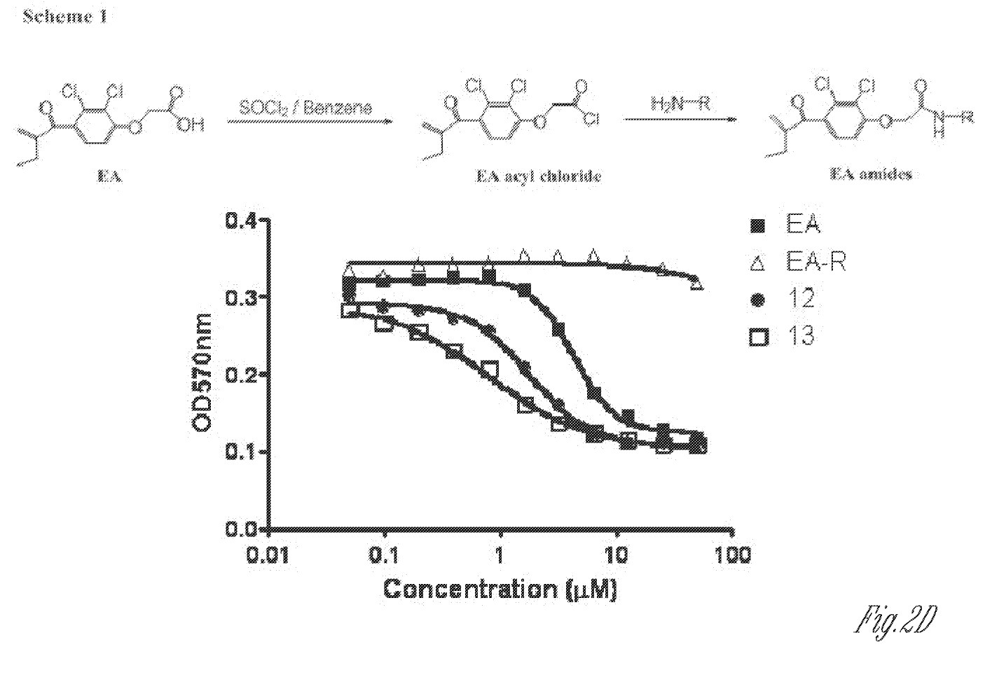

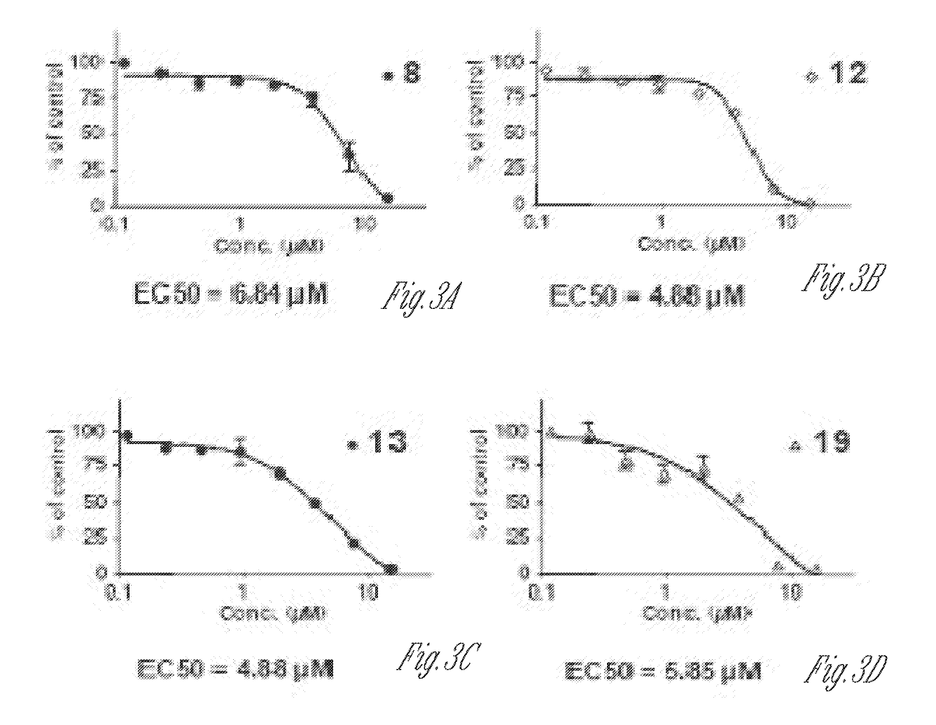

In summary, amides of EA with enhanced potency, relative to EA, toward the inhibition of Wnt signaling and of CLL cell survival were synthesized (Table 2 and FIG. 3). Differences in the potency among the various derivatives may be simply due to relative efficiency of compound delivery to cells and their ability to access the nuclear compartment and make contact with transcription factors important in Wnt signaling.

### EXAMPLE II

### Materials and Methods

### Cell Apoptosis Assays

The apoptosis of the CLL cells was determined by the analysis of mitochondrial transmembrane potential (ΔΨm) using 3,3′-dihexyloxacarbocyanine iodine (DiOC₆) and by cell membrane permeability to propidium iodide (PI). Primary CLL cells were treated with 3 μM EA, 1 mM NAC, 100 μM BHA or combined treatment as indicated. After treatment for 48 hours, the cells were stained with DiOC₆ and PI and analyzed by flow cytometry. For each assay, 100 μL of the cell culture at a density of 10⁶ cells/mL was collected at the indicated time points and transferred to polypropylene tubes containing 100 μL of 60 nM DiOC6 and 10 μg/mL PI in FACS buffer containing serum deficient RPMI medium with 0.5% bovine serum albumin (BSA). The cells were then incubated at 37° C. for 15 minutes and analyzed within 30 minutes by flow cytometry using a FACSCalibur (Becton Dickinson). Fluorescence was recorded at 525 nm (FL-1) for DiOC₆ and at 600 nm (FL-3) for PI. The apoptotic cells were determined by calculating the percentages of the DiOC₆⁺/PI⁻ CLL populations.

### RNA Isolation and Real-Time PCR

Primary CLL cells from three patients were treated with increasing amounts of EA for 16 hours. Total RNA was isolated from 1×10⁶ CLL cells by Trizol reagent (Invitrogen, Carlsbad, Calif.). The RNA samples were further purified using a Qiagen RNeasy Protect kit (Qiagen, Valencia, Calif.). The mRNA levels were quantified in duplicate by real time PCR on the iCycler iQ detection system for TaqMan assay (Bio-Rad Laboratories, Hercules, Calif.) using the following primer sets: cyclin D1 forward 5′GGCGGAGGAGAACAAACAGA3′ (SEQ ID NO:1), reverse 5′TGGCACAAGAGGCAACGA 3′ (SEQ ID NO:2) and probe 5′TCCGCAAACACGCGCAGACC 3′ (SEQ ID NO:3), Fibronectin forward 5′ACCTACGGATGACTCGTGCTTT3′ (SEQ ID NO:4), reverse 5′TTCAGACATTCGTTCCCACTCA3′ (SEQ ID NO:5) and probe 5′CCTACACAGTTTCCCATTATGCCGTTGGA 3′ (SEQ ID NO:6), Fzd5 forward 5′ CGCGAGCACAACCACATC3′ (SEQ ID NO:7), reverse 5′ AGAAGTAGACCAGGAGGAAGACGAT3′ (SEQ ID NO:8) and probe 5′ TACGAGACCACGGGCCCTGCAC3′ (SEQ ID NO:9). LEF-1 mRNA level was detected using TaqMan Gene Expression assay Hs00212390_m1 (LEF-1) (Applied Biosystems). PCR was performed using Taqman PCR Core Reagents (Applied Biosystems, Foster City, Calif., USA) according to the manufacturer's instructions. PCR cycles consisted of an initial denaturization step at 95° C. for 15 seconds and at 60° C. for 60 seconds. PCR amplification of 18S RNA was done for each sample as a control for sample loading and to allow for normalization between samples. The data were analyzed using the comparative Ct method, where Ct is the cycle number at which fluorescence first exceeds the threshold. The ΔCt values from each cell line were obtained by subtracting the values for 18S Ct from the sample Ct. One difference of Ct value represents a 2-fold difference in the level of mRNA. The mRNA level was expressed as percentage with respect to control (100%).

### Preparation of Ethacrynic Acid Antiserum

A conjugate of EA with Keyhole Limpet Hemocyanin (KLH, Sigma) was prepared by thiolation of KLH with N-succinimidyl S-acetylthioacetate (SATA), followed by allowing the SATA-KLH conjugate to form a Michael adduct with EA, as described in Hermanson (1996). Immunization of rabbits was performed by three 1 mL subcutaneous injections of approximately 0.4 mg EA-KLH conjugates. Complete Freund's adjuvant was used for the first injection. The second and third injections were performed 3 and 6 weeks after the first, using incomplete adjuvant. The rabbits were bled six weeks after the third injection for preparation of antiserum. The specificity of the antibody was confirmed by both ELISA and immunoblotting using EA conjugated to a different antigen (ovalbumin).

### Co-Immunoprecipitation and Immunoblotting

Primary CLL cells and SW480 cells were treated with the indicated amounts of EA. Cells were washed twice with PBS and resuspended in 0.5 mL lysis buffer (20 mM Tris-HCl, pH 8.0/10% glycerol/5 mM MgCl₂/0.15 M KCl/0.1% Nonidet P-40 with protease inhibitors). For CLL cells, lysates of 1 to 2×10⁷ cells were incubated with anti-LEF-1 antibody at a 1:1000 dilution overnight at 4° C., and then with saturating amounts of protein G plus/protein A agarose beads (Calbiochem) at 4° C. for 2 hours before centrifugation at 15,000 g for 5 minutes. For SW480 cells, lysates of 0.5 to 1×10⁷ cells were incubated overnight at 4° C. with saturating amounts of agarose beads linked to monoclonal antibodies specific for β-catenin (Santa Cruz Biotechnology). The beads were washed twice with lysis buffer and once with PBS. Bound proteins were eluted by boiling the samples in SDS sample buffer and resolved by SDS/PAGE followed by immunoblotting with anti-EA antibody (1:1000), anti-LEF-1 antibody (1:1000) (BD Biosciences), anti-β-catenin antibody (1:2000) (Santa Cruz Biotechnology), anti-α-catenin antibody (1:2000) (GenWay). Horseradish peroxidase-conjugated anti-IgG was used as the secondary antibody. The membranes were developed using a chemiluminescence system (ECL detection reagent, Amersham Pharmacia Life Science). For some experiments, the immunoblots were imaged with an Odyssey Infrared Imaging System (LI-COR Biosciences, Lincoln, Nebr.). The membranes were stripped with Re-Blot Western blot recycling kit (Chemicon International, Temecula, Calif.) and reprobed.

### Results

### Selective Cytotoxicity of EA to Chronic Lymphocytic Leukemia (CLL) Cells

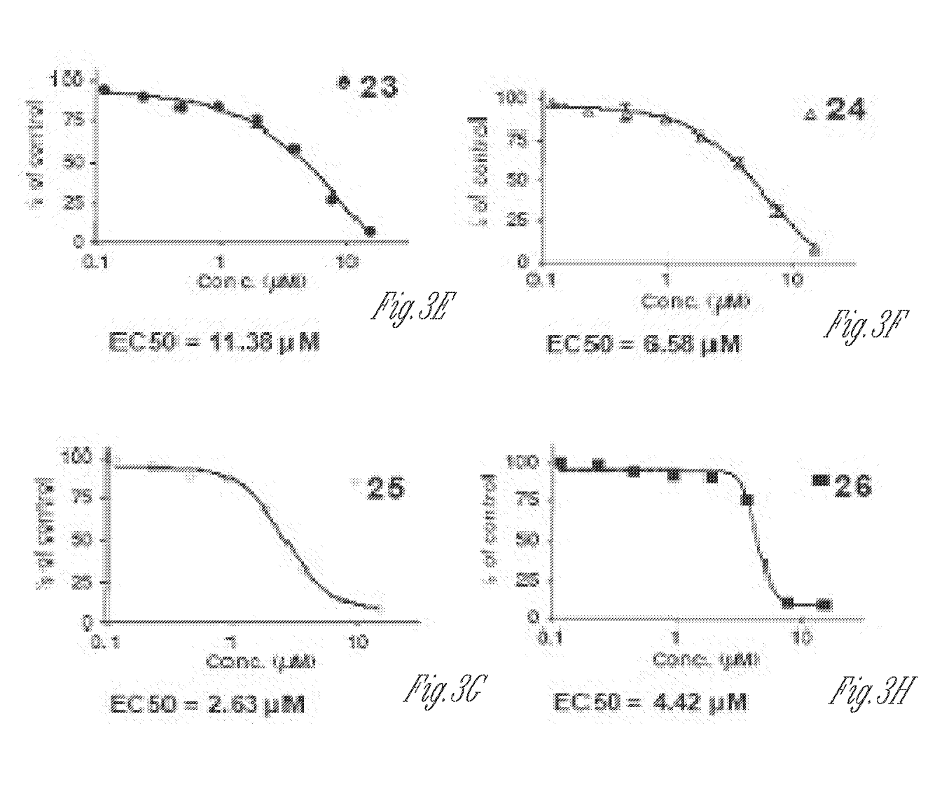

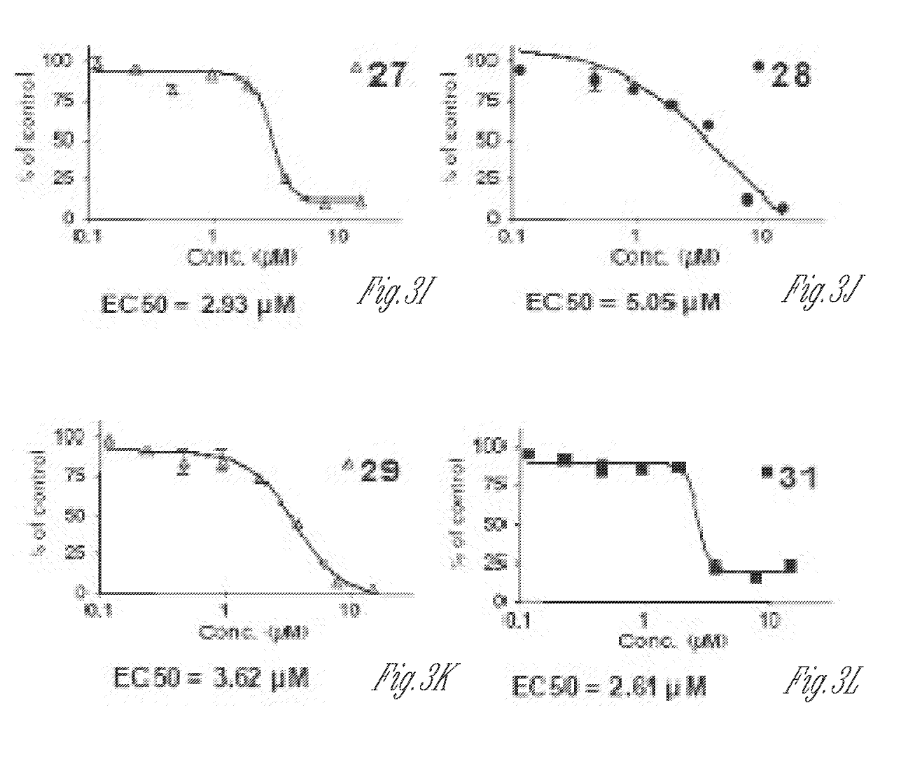

The cytotoxicity of EA was tested in different tumor cell lines, and in primary CLL cells that are known to have constitutive Wnt activation and very high levels of LEF-1. As shown in Table 3, the mean 50% inhibitory concentration (IC₅₀) of EA in these cell lines was in the 40 to 200 μM range. However, primary CLL cells are highly sensitive to EA. This drug showed selective cytotoxicity with a mean IC₅₀ of 8.56+/−3 μM in primary CLL cells, compared to 34.79+/−15.97 μM in normal peripheral blood mononuclear cells (P<0.001) (Table 3 & FIG. 4). In addition, obvious cell death occurred after treatment with EA for 48 hours (data not shown). These findings are in agreement with a previous report by Twentyman et al. (1992).

TABLE 3
Cytotoxicity of EA in different tumor cell
lines and primary CLL cells

| Cell line name | IC₅₀-EA (μM)* |
|---|---|
| Primary CLL cells | 8.56 ± 3.0 |
| Normal PBMC | 34.79 ± 15.97 |
| LNCap | 46 |
| PC3 | 67 |
| HCT116 | 58 |
| SW480 | 68 |
| HT29 | 56 |
| MCF-7 | 63 |
| SK-Mel-28 | 122 |
| HepG2 | 223 |
| A549 | 178 |
| U266 | 90 |
| B16 | 201 |
| RAMOS | 174 |

*IC₅₀ is the mean concentration of drug that reduced cell survival by 50% in at least two experiments. Primary CLL cells were isolated from nine patients. Peripheral blood mononuclear cells (PBMC) were isolated from five normal individuals.

### EA Depresses the Expression of LEF-1, Cyclin D1 and Fibronectin

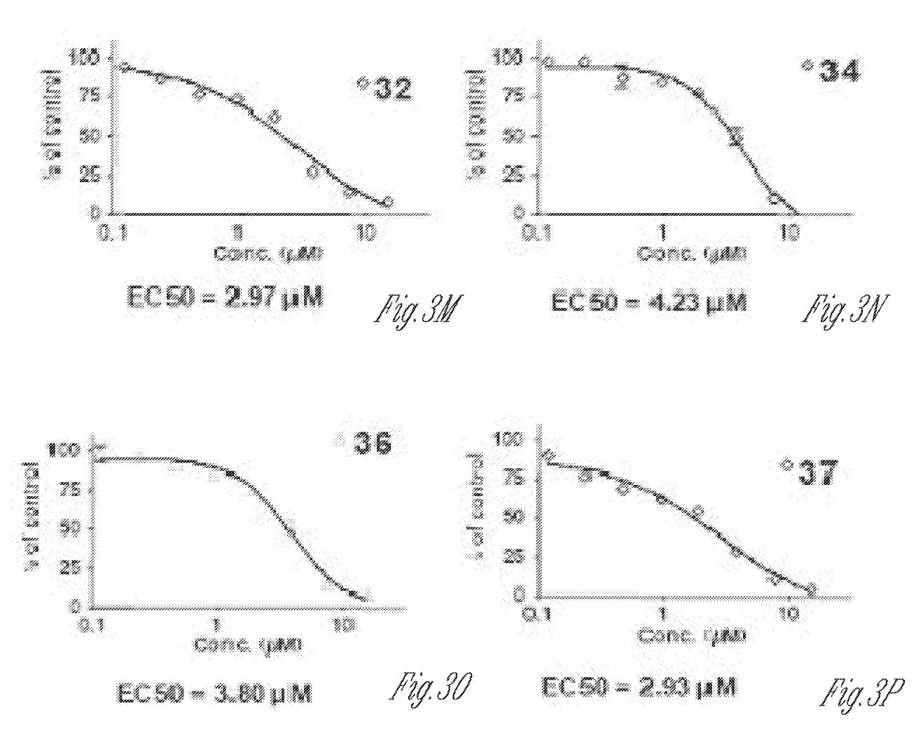

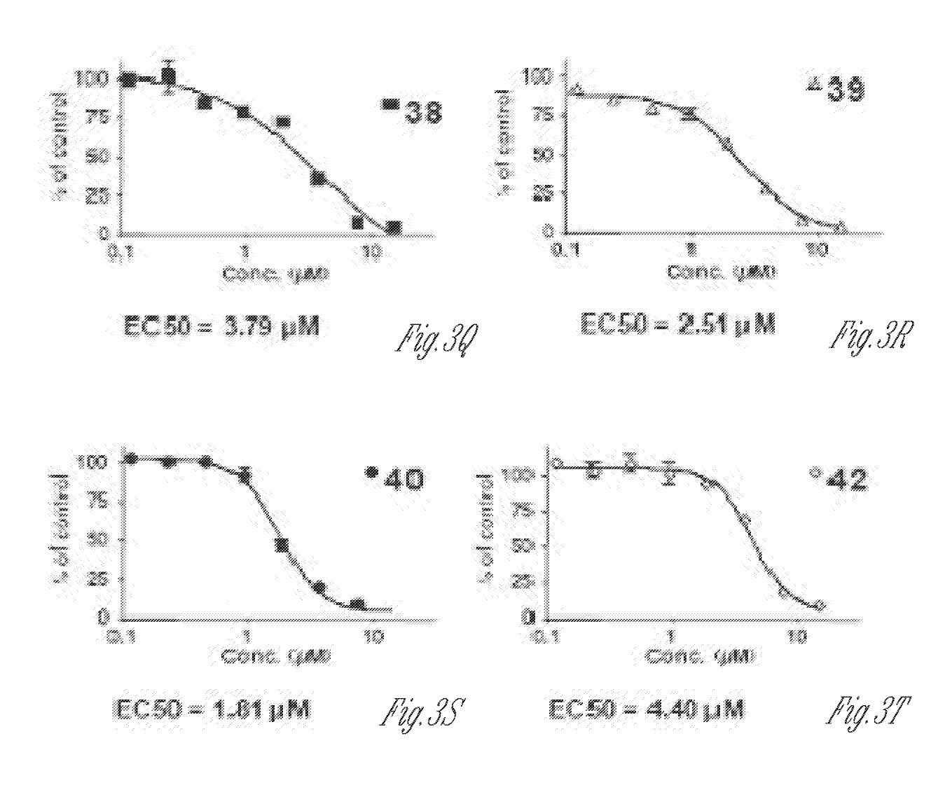

To assess the inhibitory effects of EA on Wnt/β-catenin signaling in CLL cells, real-time PCR was employed to detect the expression of some Wnt target genes. LEF-1, cyclin D1 and fibronectin are established target genes of the Wnt/β-catenin pathway (Filali et al., 2002; Gradl et al., 1999; Hovanes et al., 2001; Shtutman et al., 1999; Testu et al., 1999). The expression of Fzd5 was also detected in these experiments. Fzd5 is not a target gene of Wnt/β-catenin signaling. To determine the ability of EA to alter LEF-1, cyclin D1, fibronectin and Fzd5 transcript expression, CLL cells from three patients were treated with the drug for 16 hours, and then analyzed by real-time PCR. Total RNA input was normalized to the concentration of 18S RNA. As shown in FIG. 5, EA decreased LEF-1, cyclin D1 and fibronectin mRNA expression in a concentration-dependent fashion in CLL cells. Interestingly, EA showed dose-dependent enhancement of Fzd5 expression (FIG. 5). It is unclear how EA enhances the expression of Fzd5.

EA Directly Interacts with LEF-1 and Destabilizes the LEF-1/β-Catenin Complex

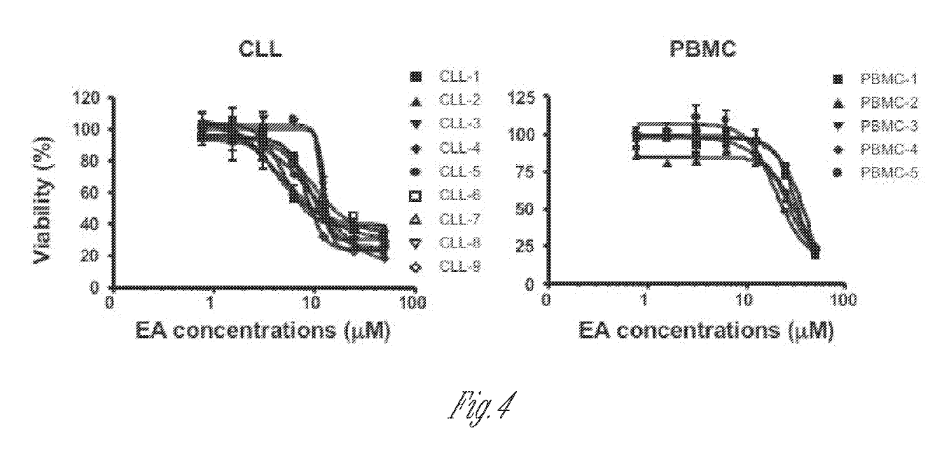

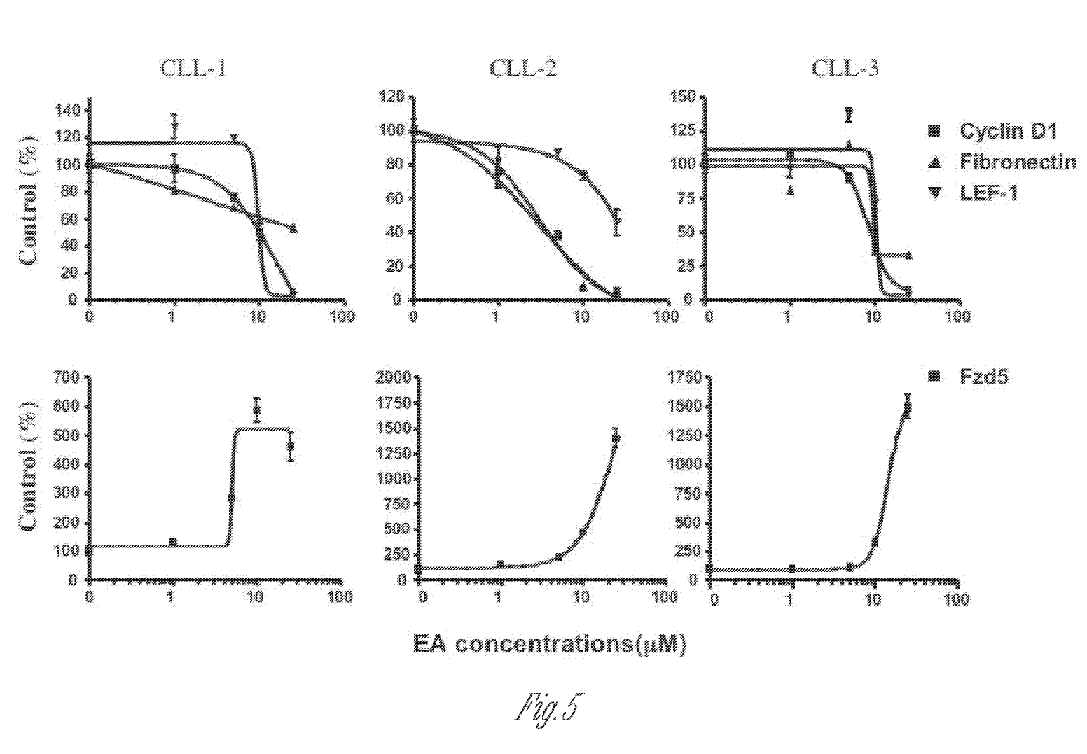

Since the initial results indicated that EA may target either β-catenin itself or its downstream factors, it was determined whether the drug could directly interact with any component of the β-catenin complex. The unsaturated ketone in EA can undergo Michael addition with free thiols. To detect such covalent modifications, an antibody to EA was conjugated to an irrelevant protein carrier, and that conjugate was used to probe CLL cells exposed to the drug by immunoblotting. As expected, EA could interact with multiple proteins in CLL extracts, but did not bind detectably to β-catenin itself (data not shown). However, the antibody to EA consistently recognized a 47 kD protein consistent with the approximate size of LEF-1 (data not shown). To determine whether EA could indeed directly interact with LEF-1, CLL cells that had been treated with the drug were lysed, immunoprecipitated with anti-LEF-1 antibody, and probed in immunoblots using the anti-EA antibody (FIG. 6A). The immunoprecipitated LEF-1 protein from CLL lysates treated with EA at 10 μM for 8 hours and 24 hours reacted strongly with the anti-EA antibody.

To test whether EA could similarly interact with LEF-1 in other tumor cells, a human colon cancer cell line SW480 was tested. It has been demonstrated that LEF-1 is constitutively associated with β-catenin in SW480 cells (Porfiri et al., 1997). If EA inhibits Wnt/β-catenin signaling through directly interacting with LEF-1, one would expect that the drug should bind to LEF-1 in the LEF-1/β-catenin complex. Hence, an anti-β-catenin antibody was used to pull down the β-catenin complex in a co-immunoprecipitation experiment. SW480 cells were treated with the indicated amounts of EA in FIG. 6 for 16 hours. Cell lysates were immunoprecipitated with anti-β-catenin antibody. The precipitated β-catenin could not be detected by anti-EA antibody (FIG. 6B). However, the anti-β-catenin antibody pulled down LEF-1 protein and EA (FIG. 6C), indicating that the drug may directly bind to LEF-1 in the LEF-1/β-catenin complex in SW480 cells.

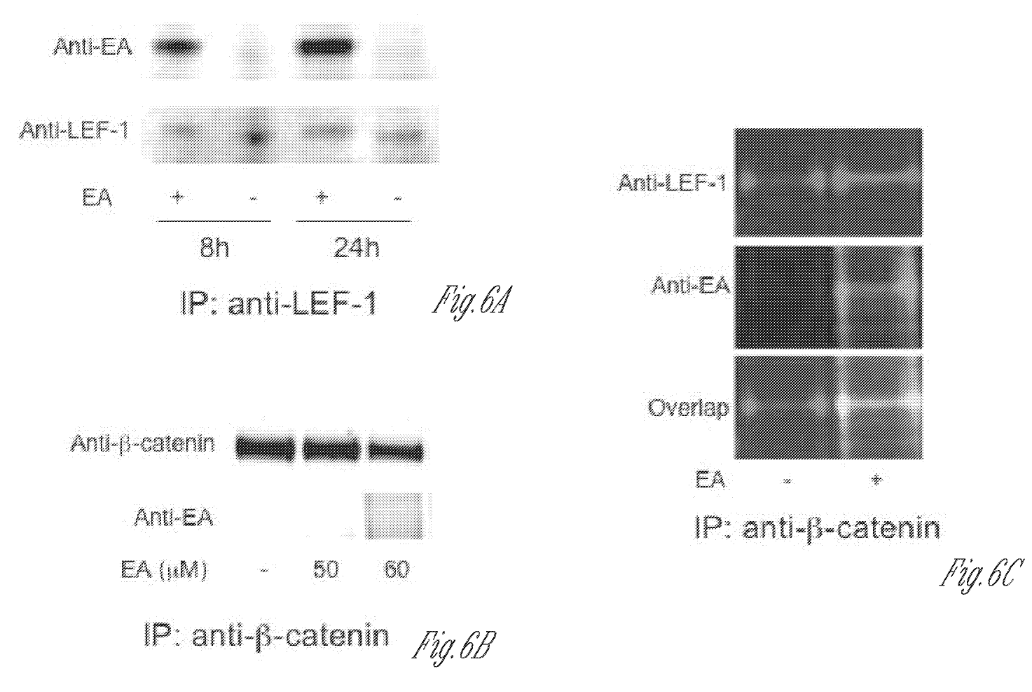

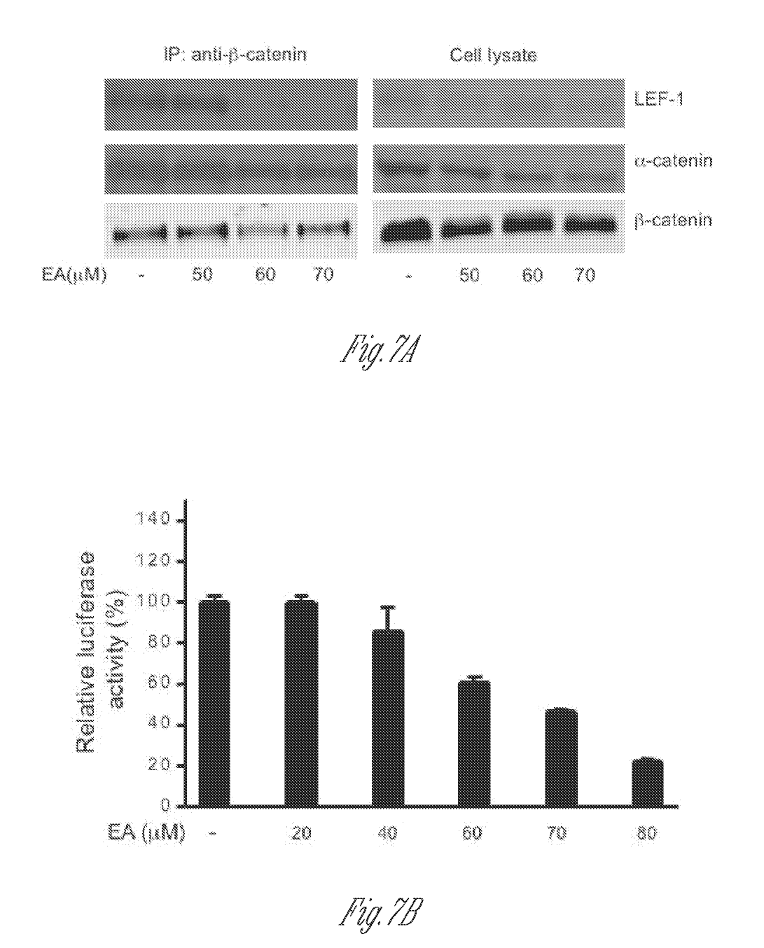

To determine the consequences of EA modification of the LEF-1/β-catenin complex, SW480 cells were treated with increasing concentrations of the drug, and after 16 hours, the β-catenin complex was pulled down using antibody against β-catenin. Immunoblot analysis demonstrated that EA decreased LEF-1 levels in the β-catenin complex in a concentration-dependent manner (FIG. 7A). However, EA had little effect on α-catenin levels. This result suggests that EA binding to LEF-1 may lead to destabilization of the LEF-1/β-catenin complex. In whole cell lysate, EA treatment decreased the level of α-catenin. It is possible that EA may downregulate the expression of α-catenin via some as yet unknown mechanism.

The effect of EA on TOPflash activity in SW480 cells was determined. EA exhibited dose-dependent inhibition at concentrations equal to and above 60 μM, the dose required to destabilize the LEF-1/β-catenin complex (FIG. 7B). This result suggests that EA may inhibit LEF-1-mediated transcription through destabilization of the LEF-1/β-catenin complex in colorectal cancer cells.

### N-acetyl-L-cysteine (NAC) Prevents EA-Mediated Effects on the Wnt/β-Catenin Pathway and on CLL Survival

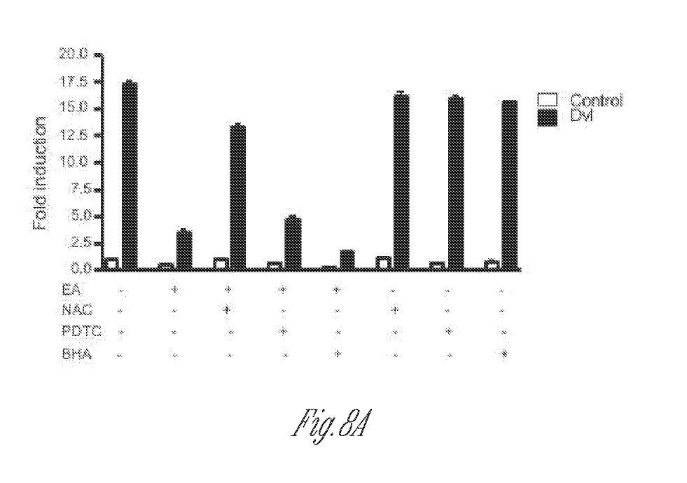

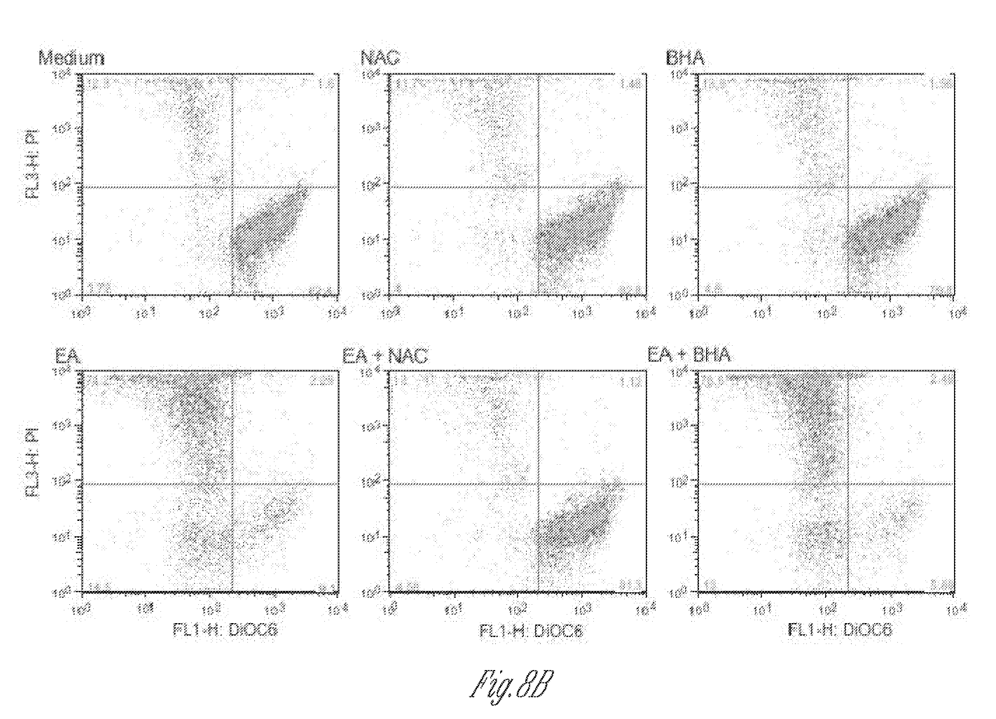

Although previous reports showed cytotoxicity of EA in different tumor cell lines, the mechanism of cell killing was unknown (Twentyman et al., 1992: Aizawa et al., 2003). To examine if the inhibition of Wnt/β-catenin signaling by EA is mediated by modulation of thiols, or by increased oxidative stress as a result of GST inhibition, cells that had been co-transfected with the TOPflash reporter and the Dvl expression plasmids were treated with EA and various antioxidants (NAC, BHA and PDTC). NAC, an antioxidant containing a reactive free thiol group, significantly prevented EA-induced inhibition of Wnt/β-catenin signaling. In contrast, neither BHA, which scavenges reactive oxygen species (ROS), but does not have a free thiol, nor PDTC, which does react efficiently with EA, did not reverse the Wnt inhibition (FIG. 8A). Consistent with this result, neither a glutathione synthesis inhibitor (buthionine sulfoximine, BSO) nor a superoxide dismutase inhibitor (2-methoxyestradiol), blocked Wnt/β-catenin signaling in the cell-based system (data not shown).

To test whether NAC has the ability to protect CLL cells from EA-induced apoptosis, a highly EA-sensitive CLL sample was treated with EA alone or combined with NAC for 48 hours. The result showed that NAC (1 mM) significantly protected CLL cells from EA-induced apoptosis, but the free radical scavenger BHA (100 μM) had no effect (FIG. 8B) suggesting that the inhibition of Wnt/β-catenin signaling and apoptosis in CLL by EA is not mediated by the generation of oxygen radicals.

### Discussion

The Wnt signaling pathway has been shown to play a critical role in the early phases of B lymphocyte development, but is thought to be less important for the survival of normal mature B cells (Reya et al., 2000). Although CLL cells have the morphological characteristics of mature B lymphocytes, they frequently over-express Wnt pathway genes associated with pro-B or pre-B cells, including Wnt3, Wnt16, the orphan Wnt receptor ROR1, and the LEF-1 transcription factor (Lu et al., 2004; Rosenwald et al., 2001; Gutierrez et al., 2007; Howe et al., 2006). The immature pro-B cells from LEF-1 deficient mice display increased sensitivity to apoptosis (Reya et al., 2000), although the exact mechanism is unclear. It was hypothesized that interference with the Wnt/β-catenin/LEF-1 pathway might sensitize CLL cells to apoptosis. The results of the present experiments support this supposition.

To identify potential pharmacologic antagonists of Wnt signaling, a 960-member library of known drugs was screened using a cell-based TOPflash reporter gene assay. Among the few drugs that blocked the Wnt reporter gene activity, at concentrations that did not affect a control reporter gene, was the loop diuretic ethacrynic acid (EA). The antagonism was not attributable to non-specific toxicity of EA. Moreover, neither DNA damaging agents nor anti-metabolites used in cancer therapy displayed inhibitory effects in this Wnt dependent system. Experiments with the cell-based reporter system demonstrated that EA inhibited Wnt/β-catenin signaling mediated not only by Wnt3a, but also by Wnt/LRP6, Dvl and β-catenin, respectively, suggesting that the drug may target either β-catenin itself or its downstream factors. Subsequent studies showed that EA could not bind to β-catenin. Instead, EA directly interacted with LEF-1, and induced the destabilization of the LEF-1/β-catenin complex. LEF-1 has been shown to have free thiol groups that are required for maintenance of its structure (Love et al., 1995). In cells whose survival depends upon LEF-1 activity, modification of these thiols by EA may be lethal.

Experiments with real-time PCR demonstrated that treatment with EA caused a dose-dependent decline in the expression of three Wnt target genes, LEF-1, cyclin D1 and fibronectin, which reflects EA inhibition of Wnt/β-catenin signaling in CLL cells. However, EA enhanced the expression of Fzd5 in a concentration-dependent manner. Fzd5 is a member of Frizzled receptor family. It has been shown to activate both canonical and noncanonical Wnt pathways through binding Wnt proteins such as Wnt5a, Wnt7a and Wnt11 (Caricasole et al., 2003; He et al., 1997; Cavodeassi et al., 2005). Interestingly, a recent study demonstrated that apoptotic agents imatinib and etoposide could also up-regulate Fzd5 expression in the myeloid cell lines K562 and HL60 (Sercan et al., 2007). The increased expression of Fzd5 might correlate with an apoptotic process.

EA is a loop diuretic drug that was formerly widely used, and demonstrated an excellent safety profile, despite its α,β-unsaturated ketone, that can modify free thiol residues of proteins. Previous studies demonstrated that reduction of the C—C double bond in EA abrogated its ability both to block Wnt signaling and to impair CLL survival (Example I). N-acetyl-L-cysteine (NAC) significantly prevented the EA-mediated inhibition of the Wnt/β-catenin pathway and EA-induced apoptosis in CLL cells. NAC is a known precursor and upregulator of GSH. It may mediate its functions by formation of GSH-conjugates that can be removed by the multidrug resistance pump or by directly reversing EA-alkylated cysteine residues. To determine whether depletion of GSH is associated with the inhibition of Wnt/β-catenin signaling, buthionine sulfoximine (BSO) was used to deplete GSH in cells. Treatment with BSO did not inhibit Wnt/β-catenin signaling (data not shown), suggesting that the depletion of GSH is probably not responsible for EA effect on Wnt/β-catenin signaling. Since EA is able to increase cellular oxidative stress through inhibiting GST, the effect of two free radical scavengers, BHA and PDTC, on EA-mediated inhibition of Wnt/β-catenin signaling, was also tested. Both scavengers could not prevent EA-induced inhibition (FIG. 8A). Moreover, no significant increase in the levels of O₂⁻ or H₂O₂ in CLL cells treated with EA was observed (results not shown). These results indicate that increased oxidative stress is not responsible for the selective killing of CLL cells by EA. Similarly, Aizawa et al. (2003) reported that EA could induce cell death in a human colon cancer cell line (DLD-1) via an oxidative stress independent mechanism.

The experiments above demonstrated potent cytoxicity of EA in primary CLL cells with IC₅₀ of 8.56+/−3 μM, while the IC₅₀ for EA inhibition of Wnt3A-induced transcription in HEK293 is about 25 μM, and the EA concentration required to destabilize the LEF-1/β-catenin complex is at least 60 μM. This difference reflects the cell type specific effect on EA sensitivity. CLL cells are known to have low levels of GSH that can react with EA (Silber et al., 1992). There is also differential dependence of cell survival on LEF-1/β-catenin signaling, which is probably critical for CLL, but not many other cell types.

Compared to cultured tumor cell lines and to normal peripheral blood mononuclear cells, primary CLL cells were 5 to 50 fold more sensitive to the cytotoxic effects of EA. It is tempting to speculate that the sensitivity may be related to the high levels in CLL of LEF-1 and its downstream effectors such as ROR1, compared to most other cell types (Gutierrez et al., 2007; Howe et al., 2006). Accordingly, LEF-1 could be a critical target for chemotherapy in CLL cells.

NF-κB signaling is another anti-apoptotic pathway which is constitutively activated in CLL cells, and may render them resistant to normal mechanisms of apoptosis (Braun et al., 2006; Furman et al., 2000). Previous studies have revealed that inhibition of NF-κB by drugs induces apoptosis of CLL cells (Furman et al., 2000; Horie et al., 2006). Han et al. (2005) reported that EA could inhibit activation of the NF-κB pathway at multiple steps. Thus, inhibition of NF-κB may synergize with Wnt antagonism to impair CLL survival.

In addition, other signaling pathways may also contribute to the cytotoxic effects of EA on various cell types. It has been reported that the mitogen activated protein kinase (MAPK) pathway may be involved in EA-induced cell death (Aizawa et al., 2003), but MAPK inhibitors are not generally cytotoxic to CLL cells. A recent study showed that EA and its butyl ester prodrug induced apoptosis in leukemia cells through a hydrogen peroxide-mediated pathway, although in CLL cells ant-oxidants other than N-acetyl-L-cysteine did not abrogate either the drug's toxicity or its ability to block Wnt signaling (Wang et al., 2007). The high sensitivity of CLL cells to EA may be attributed to its multiple effects on both Wnt and NF-κB signaling.

Recently, a significant induction of apoptosis by EA and ciclopiroxolamine (cic) in lymphoma and myeloma cells was observed (Schmidt et al., 2009). The data suggest that EA and cic can inhibit Wnt/β-catenin signalling in lymphoma and myeloma cell lines. Those results are in accordance with a recent report that the canonical Wnt signalling pathway is activated in multiple myeloma through constitutively active β-catenin (Sukhdeo et al., 2007).

In summary, these experiments suggest that EA selectively suppresses CLL survival in part due to inhibition of Wnt/β-catenin signaling. Antagonizing Wnt signaling in CLL with EA or related drugs may represent an effective treatment of this disease. O'Dwyer and colleagues reported a phase I trial of EA in patients with advanced solid tumors (Lacreta et al., 1994; O'Dwyer et al., 1991). The toxicities associated with the diuretic effect were easily managed with proper monitoring. The maximum plasma concentrations of EA ranged from 2.66 to 9.38 μg/mL (8.8 to 30.9 μM) after i.v. administration. Moreover, their results suggested that continuous i.v. infusion can be used to achieve and sustain plasma concentrations greater than 1 μg/ml (3.3 μM) for up to 3 hours (Lacreta et al., 1994). In this study, it was demonstrated the selective cytotoxicity of EA in primary CLL cells with a mean IC₅₀ of 8.56+/−3 μM, which can be achieved in patients. In addition, preliminary results have shown that treatment with 3 μM EA enhanced fludarabine-mediated apoptosis of CLL cells (data not shown). These results suggest that EA, by inhibition of the Wnt/β-catenin pathway, compromised an important survival signal in CLL cells and increased their vulnerability to cell killing induced by chemotherapeutic agents. Therefore, there is therapeutic potential for EA or derivatives thereof alone or combined with other cytotoxic agents, such as fludarabine, in CLL patients or other cancers that overexpress Wnt signaling genes, e.g., leukemias, solid tumors, or lymphomas.

### REFERENCES

Aizawa et al., Cancer Sci., 94:886 (2003).

Barker and Clevers, Nat. Rev. Drug Discovery, 5:997 (2006).

Braun et al., Cell Death Differ., 13:748 (2006).

Caligaris-Cappio and Hamblin, J. Clin. Oncol., 17: 399 (1999).

Caricasole et al., J. Biol. Chem., 278:37024 (2003).

Cavodeassi et al., Neuron, 47:43 (2005).

Clevers, Cell, 127:469 (2006).

Filali et al., J. Biol. Chem., 277:33398 (2002).

Fukuda et al., Proc. Natl. Acad. Sci. USA, 105:3047 (2008).

Furman et al., J. Immunol., 164:2200 (2000).

Gradl et al., Mol. Cell. Biol., 19:5576 (1999).

Gutierrez et al., Leukemia, 21:541 (2007).

Han et al., Shock, 23:45 (2005).

He et al., Science, 275:1652 (1997).

Hermanson, Bioconjugate Techniques. San Diego: Academic Press. pp. 467-469 (1996).

Horie et al., Leukemia, 20:800 (2006).

Hovanes et al. Nat Genet., 28:53 (2001).

Howe et al., Leuk. Res., 30:29 (2006).

Jin et al., Bioorg. Med. Chem. Lett., 19:606 (2009).

Kay et al., Hematology (Am. Soc. Hematol. Educ. Program):193 (2002).

Keating et al., Hematology (Am. Soc. Hematol. Educ. Program):153 (2003).

Kipps, T. J., In: Beuter E L M, Coller B, Kipps T J, editor. Williams Hematology. 5 ed. New York: McGraw-Hill, Inc. pp. 1017-1039 (1995).

Lacreta et al., J. Pharmacol. Exp. Ther., 270:1186 (1994).

Lambert et al., Leukemia, 6:1063 (1992).

Love et al., Nature, 376:791 (1995).

Lu et al., Proc. Natl. Acad. Sci. U.S.A., 101:3118 (2004).

Lu et al., Proc. Natl. Acad. Sci. U.S.A., 102:18567 (2005).

Moon et al., Nat. Rev. Genet., 5:691 (2004).

Nusse, Cell Res., 15: 28 (2005).

O'Dwyer et al., Cancer Res., 51:6059 (1991).

Park et al., Biochem. Biophys. Res. Commun., 331:1222 (2005).

Peifer and Polakis, Science, 287:1606-1609 (2000).

Polakis, Genes Dev., 14:1837 (2000).

Porfiri et al., Oncogene, 15:2833 (1997).

Reya et al., Immunity, 13:15 (2000).

Rosenwald et al., J. Exp. Med., 194:1639 (2001).

Rossi et al., Nature, 403:103 (2000).

Schmidt et al., Br, J. Haematol., 144:796 (2009).

Sercan et al., Oncol. Rep., 18:1563 (2007).

Shtutman et al., Proc. Natl. Acad. Sci. USA, 96:5522 (1999).

Silber et al. Blood, 80:2038 (1992).

Sukhdeo et al., Proc. Natl. Acad. Sci. USA, 104:7516 (2007).

Takahashi-Yanaga and Sasaguri, Pharmacol. Sci., 104:293 (2007).

Teo et al., Proc. Natl. Acad. Sci. U.S.A., 102:12171 (2005).

Tetsu et al., Nature, 398:422 (1999).

Tew et al., Adv. Drug Deliv. Rev., 26:91 (1997).

Tew et al., Cancer Res, 48:3622 (1988).

Twentyman et al., Leukemia, 6:726 (1992).

Wang et al., Cancer Res., 67:7856 (2007).

Willert and Jones, Genes Dev., 20:1394 (2006).

Willert et al., Nature, 423:448 (2003).

Willert, Methods Mol. Biol., 468:17 (2008).

Woltersdorf et al., J. Med. Chem., 20:1400 (1977).

Zhao et al., Bioorg. Med. Chem., 15:2701 (2007).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to inhibit or treat cancer in a mammal, comprising: administering to a mammal in need thereof an effective amount of a composition comprising an amide of ethacrynic acid.

2. The method of claim 1 wherein the cancer is a B cell cancer.

3. The method of claim 1 wherein the cancer is a solid tumor.

4. The method of claim 1 wherein the cancer is a lymphoma.

5. The method of claim 1 wherein the cancer is a leukemia.

6. The method of claim 1 wherein the cancer overexpresses one or more Wnt signaling genes.

7. The method of claim 1 wherein the cancer is a hematopoietic stem cell cancer.

8. The method of claim 7 wherein the hematopoietic cancer stem cells are Thy-1⁻, c-kit⁻, and IL-3R-alpha⁺.

9. A method to prevent, inhibit or treat an inflammatory disease or disorder associated with NF-kB in a mammal, comprising: administering to a mammal in need thereof an effective amount of a composition comprising an amide of ethacrynic acid.

10. The method of claim 1 or 9 wherein the mammal is a human.

11. The method of claim 1 or 9 wherein the amide has reduced diuretic activity relative to ethacrynic acid.

12. The method of claim 1 or 9 wherein the composition is administered intravenously.

13. The method of claim 1 or 9 wherein the composition is administered orally.

14. The method of claim 1 or 9 wherein the composition is administered in a sustained release dosage form.

15. The method of claim 1 or 9 wherein the amide has formula (I): 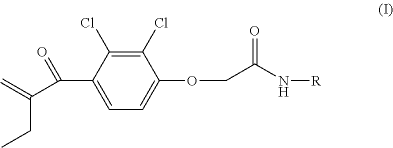
wherein R is alkyl, substituted alkyl, aryl, substituted aryl, hydroxyl, heteroaryl, substituted heteroaryl, cycloalkylalkyl, substituted cycloalkylalkyl, heterocycle or substituted heterocycle.

16. The method of claim 15 wherein the amide has formula (I): 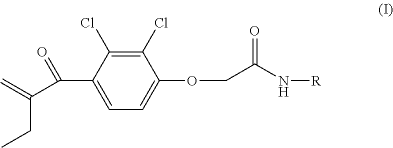
wherein R is hydroxyl or an optionally substituted alkyl, phenyl, pyridinyl, thiazolyl, naphthyl, imidizolyl, phthalazinyl, piperidinyl, or isoindolyl.

17. The method of claim 1, wherein the mammal has acute lymphoblastic leukemia (ALL), CLL or non-Hodgkin's lymphoma.

18. The method of claim 1, wherein the mammal has leukemia, lymphoma or myeloma.

19. The method of claim 1, wherein the mammal is further administered a chemotherapeutic agent.

20. The method of claim 19 wherein the chemotherapeutic agent is an alkylating agent or an anti-metabolite.

21. A compound of formula (I): 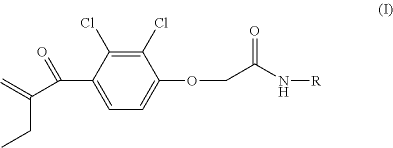
wherein R is alkyl, substituted alkyl, aryl, substituted aryl, hydroxyl, heteroaryl, substituted heteroaryl, cycloalkylalkyl, substituted cycloalkylalkyl, heterocycle or substituted heterocycle.

22. A pharmaceutical composition comprising an amide of ethacrynic acid.

23.-26. (canceled)
